SEQUENCE LISTING (10 sequences)

SEQ ID NO: 1 - protein, 119 aa, synthetic construct
  source 1..119: note = An artificially synthesized polypeptide sequence
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSS

SEQ ID NO: 2 - protein, 107 aa, synthetic construct
  source 1..107: note = An artificially synthesized polypeptide sequence
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIE

SEQ ID NO: 3 - protein, 443 aa, synthetic construct
  source 1..443: note = An artificially synthesized polypeptide sequence
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHAHYTQKSLSLSP

SEQ ID NO: 4 - protein, 214 aa, synthetic construct
  source 1..214: note = An artificially synthesized polypeptide sequence
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 6 aa, synthetic construct
  source 1..6: note = An artificially synthesized sequence
HDHAWS

SEQ ID NO: 6 - protein, 16 aa, synthetic construct
  source 1..16: note = An artificially synthesized sequence
FISYSGITNYNPSLQG

SEQ ID NO: 7 - protein, 10 aa, synthetic construct
  source 1..10: note = An artificially synthesized sequence
SLARTTAMDY

SEQ ID NO: 8 - protein, 11 aa, synthetic construct
  source 1..11: note = An artificially synthesized sequence
QASTDISSHLN

SEQ ID NO: 9 - protein, 7 aa, synthetic construct
  source 1..7: note = An artificially synthesized sequence
YGSHLLS

SEQ ID NO: 10 - protein, 9 aa, synthetic construct
  source 1..9: note = An artificially synthesized sequence
GQGNRLPYT

(19) United States
(12) Patent Application Publication
(10) Pub. No.: US 2025/0127891 A1
(43) Pub. Date: Apr. 24, 2025

(54) TREATMENT OF AUTOIMMUNE ENCEPHALITIS WITH SATRALIZUMAB

(71) Applicants: Chugai Seiyaku Kabushiki Kaisha, Tokyo (JP); Hoffmann-La Roche Inc., Little Falls, NJ (US); Genentech, Inc., South San Francisco, CA (US)

(72) Inventors: Takatoshi Ozawa, Tokyo (JP); Hiroaki Ida, Tokyo (JP); Hajime Ito, Tokyo (JP); Shunsuke Yoshida, Tokyo (JP); Jillian Smith, Welwyn Garden City (GB); Sian Lennon-Chrimes, Welwyn Garden City (GB); Gaelle Klingelschmitt, Basel (CH); Hans-Christian Von Buedingen, Basel (CH); Hanna Silber Baumann, Basel (CH); James Overell, Basel (CH); Sharmila Rajan, South San Francisco, CA (US)

(21) Appl. No.: 18/729,273

(22) PCT Filed: Jan. 18, 2023
(86) PCT No.: PCT/JP2023/001270

§ 371 Date: Jul. 16, 2024

Related U.S. Application Data
(60) Provisional application No. 63/300,893, filed on Jan. 19, 2022.

(51) Int. Cl. A61K 39/395; A61K 31/52; A61K 31/5377; A61K 31/675; A61K 39/00; A61K 45/06

(52) U.S. Cl. CPC A61K 39/3955; A61K 31/52; A61K 31/5377; A61K 31/675; A61K 45/06; A61K 2039/505; A61K 2039/54; A61K 2039/545

(57) ABSTRACT

All currently available treatment options for autoimmune encephalitis such as anti-NMDAR encephalitis and anti-LGI1 encephalitis carry substantial potential safety risks. Further, no approved therapies exist for anti-NMDAR encephalitis and anti-LGI1 encephalitis. The invention provides a means for a treatment for autoimmune encephalitis (AIE) such as NMDAR encephalitis and anti-LGI1 encephalitis, comprising an IL-6 inhibitor such as anti-IL-6 receptor antibody or antigen binding fragment thereof.

### TECHNICAL FIELD

The present invention relates to a medicament or a pharmaceutical composition for treatment, or for reducing the risk of relapse, of, autoimmune encephalitis (also referred as AIE or AE), including but not limited to, anti-N-methyl-D-aspartic acid receptor (NMDAR) encephalitis and anti-leucine-rich glioma-inactivated 1 (LGI1) encephalitis, comprising an anti-IL-6 receptor antibody or antigen binding fragment thereof. The present invention also relates to a method of treatment, or of reducing the risk of relapse, of said autoimmune encephalitis (AIE), including but not limited to, anti-N-methyl-D-aspartic acid receptor (NMDAR) encephalitis and anti-leucine-rich glioma-inactivated 1 (LGI1) encephalitis, by administering an anti-IL-6 receptor antibody or antigen binding fragment thereof to a subject in need thereof.

### BACKGROUND ART

Acute encephalitis is a rare and debilitating neurological disorder that develops in patients of all ages, presenting as a rapidly progressive encephalopathy as a consequence of brain inflammation [NPL 1]. Autoimmune encephalitis (AIE) includes disorders that are associated with an identifiable etiological driver, usually a tumor, and disorders that are regarded as idiopathic. Paraneoplastic immune-mediated encephalitis syndromes may be associated with antibodies against intracellular neuronal proteins (onconeuronal proteins such as anti-Hu). These are of unclear pathogenic significance and tend to denote syndromes that respond poorly to immunotherapy.

The identification of antibodies against neuronal cell surface and synaptic proteins, which are thought to be directly pathogenic, delineates a population with better response to immunotherapies in general, whether an associated tumor is identified or not. Among these AIE subtypes, NMDAR- and LGI1-mediated encephalitis represent two of the most common [NPL 2] and best characterized syndromes.

NMDAR encephalitis is an immune-mediated disease characterized by a complex neuropsychiatric syndrome, which includes cognitive impairment and seizures, and the presence of cerebral spinal fluid (CSF) antibodies against the GluN1 subunit of the NMDAR. Using mouse models, these antibodies were shown to crosslink NMDARs, altering their surface dynamics and interaction with other synaptic proteins, and causing their internalization along with severe impairment of synaptic plasticity and NMDAR network function [NPL 3-6]. NMDAR encephalitis has an estimated incidence of 1.5 per million population per year, occurs mostly in females (a female to male ratio of approximately 8:2), and has a median age of incidence of 21 years (age range: <1-85 years). Patients develop a constellation of symptoms that varies according to the stage of the disease and that clinically suggests the diagnosis [NPL 7].

LGI1 encephalitis is an immune-mediated disease in which most patients present with limbic encephalitis, characterized by a subacute disturbance of memory and behavior, often accompanied by seizures. It is associated with antibodies directed to LGI1 protein. LGI1 encephalitis is more common in middle-aged and elderly males (over 50 years of age) [NPL 8]. Most LGI1 protein is expressed in the hippocampus and broader medial temporal lobe, from where it is secreted into the synaptic space. LGI1 is part of an inhibitory pathway linking the presynaptic voltage-gated K+ channel (VGKC) and the postsynaptic a-amino-3-hydroxy-5-methyl-4-isoxazolepropionic acid receptor (AMPAR) [NPL 9 and NPL 10]. LGI1 encephalitis has an estimated prevalence of 7 per million population [NPL 11], and estimated incidence of 0.83 per 1 million individuals [NPL 12].

No approved therapies exist for NMDAR or LGI1 encephalitis. The treatment approach to NMDAR and LGI1 encephalitis initially involves first-line therapies (e.g., steroids, intravenous immunoglobulins [IVIG], or plasma exchange), as detailed in a recent survey conducted by the Autoimmune Encephalitis Alliance Clinicians Network [NPL 13]. High-dose steroids are most commonly employed, but the distribution of first-line treatments in the two most common disorders is very similar, as illustrated below.

Escalation principles, second-line therapy utilization, and the specific second-line therapies used are similar between the two disorders, although steroid responsiveness is more commonly seen in LGI1 encephalitis [NPL 14], and the long-term use of steroids is more commonly employed in this disorder.

Although a majority of patients with cell surface antibody-positive disease achieve a stable condition with currently used treatment options, no prospective randomized trials have been reported to date in this population, and all therapeutics are used based on anecdotal evidence. Several limitations apply to the current AIE treatment paradigm that highlight remaining critical unmet needs for treatments with rapid and demonstrated effects on long-term efficacy and safety. For example, not all patients respond to these medications, and deficiencies remain, including cognitive deficits, insufficient seizure resolution, dependence on high-dose corticosteroids, and a need for durable, faster acting agents. There is an urgent need for prospectively generated evidence in AIE to guide treatment choice to address both acute as well as long-term effects of this rare neurological disorder.

Humanized antibodies like tocilizumab are first-generation antibody drugs. By improving first-generation antibody drugs, second-generation antibody drugs with improved efficacy, convenience, and cost are being developed [PTL 2 and PTL 3]. Among the second-generation antibody drugs is satralizumab (SA237), which is a novel anti-IL-6 receptor antibody to which improvement technologies such as enhancement of antigen-binding ability, pharmacokinetics, and stability, and reduction of immunogenicity risk, have been applied [PTL 3 and PTL 4].

Satralizumab is a humanized anti-IL-6 receptor monoclonal antibody with pH-dependent antigen binding. It specifically targets the human IL-6 receptor (IL-6R) and suppresses IL-6 signaling by inhibiting the binding of IL-6 to membrane-bound IL-6R and soluble IL-6R. Satralizumab was constructed by modifying the amino acid sequence of tocilizumab to prolong its plasma half-life. Satralizumab also shows a decreased antibody molecule isoelectric point and stronger binding to FcRn compared to tocilizumab. Moreover, its Fc region has been modified to minimize the antibody-dependent cellular cytotoxicity and complement-dependent cytotoxic effector activity compared to tocilizumab.

Prior-art literature information related to the invention of the present application is shown below.

Satralizumab has demonstrated efficacy and safety and is indicated for another autoantibody-mediated disease (i.e., NMOSD) as monotherapy or as an add-on to immunosuppressive therapy (IST; i.e., oral corticosteroid [OCS], azathioprine, or mycophenolate mofetil). The double-blind period of the Phase III studies in NMOSD included a total of 178 participants. Of the 178 participants, 104 participants were treated with a satralizumab dose of 120 mg subcutaneously every 4 weeks (Q4W) and 74 participants with placebo. Overall, satralizumab as monotherapy or in combination with IST was well tolerated by participants with NMOSD.

All currently available treatment options for AIE (including high-dose corticosteroids and cyclophosphamide) carry substantial potential safety risks. There is no evidence from randomized controlled trials to support treatment decisions. In addition, not all patients respond to currently used medications, and deficiencies remain, including cognitive deficits, insufficient seizure resolution, dependence on high-dose corticosteroids and a need for durable, faster acting agents.

### CITATION LIST

### Patent Literature

[PTL 1] US2012/0039840
[PTL 2] WO2009/041621
[PTL 3] WO2010/035769
[PTL 4] WO2016/136933

### Non-Patent Literature

[NPL 1] Venkatesan A, Tunkel A R, Bloch K C, et al., Clin Infect Dis 2013; 57:1114-28
[NPL 2] Leypoldt F, Wandinger K-P, Bien C, et al., Eur Neurol Rev 2013; 8:31-7
[NPL 3] Hughes E G, Peng X, Gleichman A J, et al., J Neurosci 2010; 30:5866-75
[NPL 4] Mikasova L, De Rossi P, Bouchet D, et al., Brain 2012; 135:1606-21
[NPL 5] Moscato E H, Peng X, Jain A, et al., Ann Neurol 2014; 76:108-19
[NPL 6] Rosch R E, Wright S, Cooray G, et al., Proc Natl Acad Sci USA 2018; 115:E9916-25
[NPL 7] Dalmau J, Armangue T, Lancet Neurology 2019; 11:1045-1057
[NPL 8] Hang H L, Zhang J H, Chen D W, et al., Front Neurol 2020; 11:852
[NPL 9] Petit-Pedrol M, Sell J, Planaguma J, et al., Brain 2018; 141:3144-59
[NPL 10] Ohkawa T, Fukata Y, Miwako Y, et al., J Neurosci 2013; 33:18161-74
[NPL 11] Dubey D, Pittock S, Kelly C, et al., Ann Neurol 2018; 83:166-77
[NPL 12] van Sonderen A, Thijs R, Coenders E, et al., Neurology 2016; 87:1449-56
[NPL 13] Abboud H, Probasco J, Irani S R, et al., J Neurol Neurosurg Psychiatry 2021. doi: 10.1136/jnnp-2020-325302
[NPL 14] Gadoth A, Pittock S, Dubey D, et al., Ann Neurol. 2017; 82:79-92

### SUMMARY OF INVENTION

### Technical Problem

Current treatment of autoimmune encephalitis (AIE) such as anti-N-methyl-D-aspartic acid receptor (NMDAR) encephalitis and anti-leucine-rich glioma-inactivated 1 (LGI1) encephalitis exclusively use off-label immune therapies and is based on expert opinion, retrospective case series, and open-label studies. All currently available treatment options for AIE (including high-dose corticosteroids and cyclophosphamide) carry substantial potential safety risks. Further, no approved therapies exist for anti-NMDAR encephalitis and anti-LGI1 encephalitis. Several unmet needs exist, including the frequent occurrence of long-term cognitive deficits, insufficient seizure control, frequent dependence on high-dose corticosteroids, and faster acting but durable immunotherapy. There is a need for prospectively generated evidence-based treatments to meaningfully lessen the acute and long-term con-sequences of these disorders.

### Solution to Problem

To solve the above-mentioned problem, the inventors designed a phase III, randomized, double-blind (DB), placebo-controlled, multicenter basket study to evaluate the efficacy, safety, pharmacokinetics, and pharmacodynamics of satralizumab compared with placebo for the treatment of anti-NMDAR encephalitis and anti-LGI1 encephalitis. The study demonstrates that satralizumab addresses the above noted unmet needs, and also establishes the effectiveness of satralizumab in patients with NMDAR or LGI1 encephalitis for treating AIE, preventing relapses of the AIE, and reducing a risk of relapse of the AIE.

The present disclosure includes but not limited to the embodiments as exemplarily described below.
[A1.1] A medicament for treating or reducing risk of relapse of an autoimmune encephalitis (AIE) in a subject, comprising an IL-6 inhibitor as an active ingredient.
[A1.2] The medicament of A1.1, wherein the IL-6 inhibitor is an anti-IL-6 antibody or antigen-binding fragment thereof, or an anti-IL-6 receptor antibody or antigen binding fragment thereof.
[A1.3] The medicament of A1.1 or A1.2, wherein the IL-6 inhibitor is an anti-IL-6 receptor antibody or antigen binding fragment thereof.
[A1.4] The medicament of any one of A1.1-A1.3, wherein the IL-6 inhibitor is a humanized antibody.
[A1.5] The medicament of any one of A1.1-A1.4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.
[A1.6] The medicament of A1.5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.
[A1.7] The medicament of A1.5 or A1.6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.
[A1.8] The medicament of any one of A1.5-A1.7, wherein the IL-6 inhibitor is satralizumab.
[A1.9] The medicament of any one of A1.1-A1.8, for delaying relapse of, reducing frequency of relapse of, reducing severity of relapse of, or reducing the need for rescue therapy for relapse of the autoimmune encephalitis in the subject.
[A1.10] The medicament of any one of A1.1-A1.9, wherein the subject is positive for anti-N-methyl-D-aspartic acid receptor (NMDAR) antibody or anti-leucine rich glioma-inactivated 1 (LGI1) antibody.
[A1.11] The medicament of any one of A1.1-A1.10, wherein the autoimmune encephalitis is a disease other than Bickerstaff's brainstem encephalitis, acute disseminated encephalomyelitis, Hashimoto's encephalopathy, primary angiitis of the central nervous system (CNS), and Rasmussen's encephalitis.
[A1.12] The medicament of any one of A1.1-A1.11, wherein an anti-NMDAR antibody is detected in the cerebrospinal fluid (CSF) from the subject using a cell-based assay.
[A1.13] The medicament of any one of A1.1-A1.12, wherein the autoimmune encephalitis is anti-NMDAR encephalitis.
[A1.14] The medicament of any one of A1.1-A1.11, wherein an anti-LGI1 antibody is detected in the serum or cerebrospinal fluid (CSF) from the subject using a cell-based assay.
[A1.15] The medicament of A1.14, wherein the subject has experienced working memory deficits, seizures, or psychiatric symptoms suggesting involvement of the limbic system.
[A1.16] The medicament of any one of A1.1-A1.11 and A1.14-A1.15, the autoimmune encephalitis is anti-LGI1 encephalitis.
[A1.17] The medicament of any one of A1.1-A1.16, wherein the autoimmune encephalitis is an anti-NMDAR or anti-LGI1 encephalitis.
[A1.18] The medicament of any one of A1.1-A1.17, wherein the subject is negative for anti-caspr2, anti-IgLON5, anti-DPPX, anti-GABA^{A}, anti-neurexin-3 alpha, and antimyelin oligodendrocyte glycoprotein (MOG) antibodies.
[A1.19] The medicament of any one of A1.1-A1.18, wherein the subject is negative for cell surface neuronal antibodies or glial antibodies other than anti-NMDAR and anti-LGI1 antibodies.
[A1.20] The medicament of any one of A1.1-A1.19, wherein the subject is (i) anti-NMDAR antibody-positive and aged 12 years or older; or (ii) anti-LGI1 antibody-positive and aged 18 years or older.
[A1.21] The medicament of any one of A1.1-A1.20, wherein the subject is receiving no ongoing chronic immunosuppressive therapy.
[A1.22] The medicament of any one of A1.1-A1.20, wherein the subject is receiving ongoing treatment with a stable dose of azathioprine (AZA), mycophenolate mofetil (MMF), intravenous (IV) cyclophosphamide, oral corticosteroid (OCS), or a combination of AZA or MMF or IV cyclophosphamide and OCS.
[A1.23] The medicament of any one of A1.5-A1.22, which is characterized in that the medicament is used such that 60 mg or 120 mg, 120 mg or 180 mg, and 180 mg or 240 mg of the anti-IL-6 receptor antibody or antigen binding fragment thereof is administered to the subject with body weight of less than 40 kg, between 40 and 100 kg, and over 100 kg respectively for each administration.
[A1.24] The medicament of any one of A1.5-A1.22, which is characterized in that the medicament is used such that 60 mg of the anti-IL-6 receptor antibody or antigen binding fragment thereof is administered to the subject with body weight of less than 40 kg for each administration.
[A1.25] The medicament of any one of A1.5-A1.22, which is characterized in that the medicament is used such that 120 mg of the anti-IL-6 receptor antibody or antigen binding fragment thereof is administered to the subject with body weight of less than 40 kg for each administration.
[A1.26] The medicament of any one of A1.5-A1.22, which is characterized in that the medicament is used such that 120 mg of the anti-IL-6 receptor antibody or antigen binding fragment thereof is administered to the subject with body weight of between 40 and 100 kg for each administration.
[A1.27] The medicament of any one of A1.5-A1.22, which is characterized in that the medicament is used such that 180 mg of the anti-IL-6 receptor antibody or antigen binding fragment thereof is administered to the subject with body weight of between 40 and 100 kg for each administration.
[A1.28] The medicament of any one of A1.5-A1.22, which is characterized in that the medicament is used such that 180 mg of the anti-IL-6 receptor antibody or antigen binding fragment thereof is administered to the subject with body weight of over 100 kg for each administration.
[A1.29] The medicament of any one of A1.5-A1.22, which is characterized in that the medicament is used such that 240 mg of the anti-IL-6 receptor antibody or antigen binding fragment thereof is administered to the subject with body weight of over 100 kg for each administration.
[A1.30] The medicament of any one of A1.5-A1.29, which is characterized in that the medicament is used such that the anti-IL-6 receptor antibody or antigen binding fragment thereof is administered to the subject subcutaneously.
[A1.31] The medicament of any one of A1.5-A1.30, which is characterized in that the medicament is used such that the anti-IL-6 receptor antibody or antigen binding fragment thereof is administered to the subject every two weeks (Q2W) for three times, and thereafter every four weeks (Q4W).
[A1.32] The medicament of any one of A1.1-A1.31, which is characterized in that the medicament is used in combination with an immunosuppressive therapy (IST).
[A1.33] The medicament of A1.32, wherein the IST is a therapy with (i) an immunosuppressive agent selected from the group consisting of azathioprine (AZA), mycophenolate mofetil (MMF), and intravenous (IV) cyclophosphamide; (ii) oral corticosteroid (OCS); or (iii) a combination of (i) and (ii).
[A1.34] The medicament of A1.33, wherein the immunosuppressive agent comprises prednisone, prednisolone or equivalent thereof.
[A1.35] The medicament of any one of A1.1-A1.34, which improving the modified Rankin Scale (mRS) score.
[A1.36] The medicament of A1.35, which
   reduces the time to the modified Rankin Scale (mRS) score improvement without use of rescue therapy
   delays the time to rescue therapy
   delays the time to seizure freedom or cessation of status epilepticus without use of rescue therapy
   improves the Clinical Assessment Scale of Encephalitis (CASE) score
   improves the Montreal Cognitive Assessment (MOCA) total score
   improves the Rey Auditory Verbal Learning Test (RAVLT) score for the subject with an anti-LGI1 antibody and/or
   improves the mRS score for the subject with an anti-NMDAR antibody.
[A1.37] The medicament of any one of A1.1-A1.36, which improves the subject's scores of one or more of the followings: modified Rankin Scale (mRS), Clinical Assessment Scale of Encephalitis (CASE), Montreal Cognitive Assessment (MOCA), Rey Auditory Verbal Learning Test (RAVLT), or Columbia-Suicide Severity Rating Scale (C-SSRS).
[A1.38] The medicament of any one of A1.1-A1.36, which reduces the subject's modified Rankin Scale (mRS) score, or Clinical Assessment Scale of Encephalitis (CASE) score; or increases the subject's Montreal Cognitive Assessment (MOCA) total score, or Rey Auditory Verbal Learning Test (RAVLT) score.
[A1.39] The medicament of any one of A1.1-A1.38, which is administered by a subcutaneous administration device.
[A1.40] The medicament of A1.39, wherein the the subcutaneous administration device is a pre-filled syringe (PFS) optionally comprising a needle safety device (PFS-NSD) or autoinjector (AI).
[A2.1] A pharmaceutical composition for treating or reducing risk of relapse of an autoimmune encephalitis (AIE) in a subject, comprising an IL-6 inhibitor as an active ingredient.
[A2.2] The pharmaceutical composition of A2.1, wherein the IL-6 inhibitor is an anti-IL-6 antibody or antigen-binding fragment thereof, or an anti-IL-6 receptor antibody or antigen binding fragment thereof.
[A2.3] The pharmaceutical composition of A2.1 or A2.2, wherein the IL-6 inhibitor is an anti-IL-6 receptor antibody or antigen binding fragment thereof.
[A2.4] The pharmaceutical composition of any one of A2.1-A2.3, wherein the IL-6 inhibitor is a humanized antibody.
[A2.5] The pharmaceutical composition of any one of A2.1-A2.4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.
[A2.6] The pharmaceutical composition of A2.5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.
[A2.7] The pharmaceutical composition of A2.5 or A2.6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.
[A2.8] The pharmaceutical composition of any one of A2.5-A2.7, wherein the IL-6 inhibitor is satralizumab.
[A2.9] The pharmaceutical composition of any one of A2.1-A2.8, for delaying relapse of, reducing frequency of relapse of, reducing severity of relapse of, or reducing the need for rescue therapy for relapse of the autoimmune encephalitis in the subject.
[A2.10] The pharmaceutical composition of any one of A2.1-A2.9, wherein the subject is positive for anti-N methyl-D aspartic acid receptor (NMDAR) antibody or anti-leucine rich glioma inactivated 1 (LGI1) antibody.
[A2.11] The pharmaceutical composition of any one of A2.1-A2.10, wherein the autoimmune encephalitis is a disease other than Bickerstaff's brainstem encephalitis, acute disseminated encephalomyelitis, Hashimoto's encephalopathy, primary angiitis of the central nervous system (CNS), and Rasmussen's encephalitis.
[A2.12] The pharmaceutical composition of any one of A2.1-A2.11, wherein an anti-NMDAR antibody is detected in the cerebral spinal fluid (CSF) from the subject using a cell-based assay.
[A2.13] The pharmaceutical composition of any one of A2.1-A2.12, wherein the autoimmune encephalitis is anti-NMDAR encephalitis.
[A2.14] The pharmaceutical composition of any one of A2.1-A2.11, wherein an anti-LGI1 antibody is detected in the serum or cerebral spinal fluid (CSF) from the subject using a cell-based assay.
[A2.15] The pharmaceutical composition of A2.14, wherein the subject has experienced working memory deficits, seizures, or psychiatric symptoms suggesting involvement of the limbic system.
[A2.16] The pharmaceutical composition of any one of A2.1-A2.11 and A2.14-A2.15, the autoimmune encephalitis is anti-LGI1 encephalitis.
[A2.17] The pharmaceutical composition of any one of A2.1-A2.16, wherein the autoimmune encephalitis is an anti-NMDAR or anti-LGI1 encephalitis.
[A2.18] The pharmaceutical composition of any one of A2.1-A2.17, wherein the subject is negative for anti-caspr2, anti-IgLON5, anti-DPPX, anti-GABA^{A}, anti-neurexin-3 alpha, and anti-myelin oligodendrocyte glycoprotein (MOG) antibodies.
[A2.19] The pharmaceutical composition of any one of A2.1-A2.18, wherein the subject is negative for cell surface neuronal antibodies or glial antibodies other than anti-NMDAR and anti-LGI1 antibodies.
[A2.20] The pharmaceutical composition of any one of A2.1-A2.19, wherein the subject is (i) anti-NMDAR antibody-positive and aged 12 years or older; or (ii) anti-LGI1 antibody-positive and aged 18 years or older.
[A2.21] The pharmaceutical composition of any one of A2.1-A2.20, wherein the subject is receiving no ongoing chronic immunosuppressive therapy.
[A2.22] The pharmaceutical composition of any one of A2.1-A2.20, wherein the subject is receiving ongoing treatment with a stable dose of azathioprine (AZA), mycophenolate mofetil (MMF), intravenous (IV) cyclophosphamide, oral corticosteroid (OCS), or a combination of AZA or MMF or IV cyclophosphamide and OCS.
[A2.23] The pharmaceutical composition of any one of A2.5-A2.22, which is characterized in that the pharmaceutical composition is used such that 60 mg or 120 mg, 120 mg or 180 mg, and 180 mg or 240 mg of the anti-IL-6 receptor antibody or antigen binding fragment thereof is administered to the subject with body weight of less than 40 kg, between 40 and 100 kg, and over 100 kg respectively for each administration.
[A2.24] The pharmaceutical composition of any one of A2.5-A2.22, which is characterized in that the pharmaceutical composition is used such that 60 mg of the anti-IL-6 receptor antibody or antigen binding fragment thereof is administered to the subject with body weight of less than 40 kg for each administration.
[A2.25] The pharmaceutical composition of any one of A2.5-A2.22, which is characterized in that the pharmaceutical composition is used such that 120 mg of the anti-IL-6 receptor antibody or antigen binding fragment thereof is administered to the subject with body weight of less than 40 kg for each administration.
[A2.26] The pharmaceutical composition of any one of A2.5-A2.22, which is characterized in that the pharmaceutical composition is used such that 120 mg of the anti-IL-6 receptor antibody or antigen binding fragment thereof is administered to the subject with body weight of between 40 and 100 kg for each administration.
[A2.27] The pharmaceutical composition of any one of A2.5-A2.22, which is characterized in that the pharmaceutical composition is used such that 180 mg of the anti-IL-6 receptor antibody or antigen binding fragment thereof is administered to the subject with body weight of between 40 and 100 kg for each administration.
[A2.28] The pharmaceutical composition of any one of A2.5-A2.22, which is characterized in that the pharmaceutical composition is used such that 180 mg of the anti-IL-6 receptor antibody or antigen binding fragment thereof is administered to the subject with body weight of over 100 kg for each administration.
[A2.29] The pharmaceutical composition of any one of A2.5-A2.22, which is characterized in that the pharmaceutical composition is used such that 240 mg of the anti-IL-6 receptor antibody or antigen binding fragment thereof is administered to the subject with body weight of over 100 kg for each administration.
[A2.30] The pharmaceutical composition of any one of A2.5-A2.29, which is characterized in that the pharmaceutical composition is used such that the anti-IL-6 receptor antibody or antigen binding fragment thereof is administered to the subject subcutaneously.
[A2.31] The pharmaceutical composition of any one of A2.5-A2.30, which is characterized in that the pharmaceutical composition is used such that the anti-IL-6 receptor antibody or antigen binding fragment thereof is administered to the subject every two weeks (Q2W) for three times, and thereafter every four weeks (Q4W).
[A2.32] The pharmaceutical composition of any one of A2.1-A2.31, which is characterized in that the pharmaceutical composition is used in combination with an immunosuppressive therapy (IST).
[A2.33] The pharmaceutical composition of A2.32, wherein the IST is a therapy with (i) an immunosuppressive agent selected from the group consisting of azathioprine (AZA), mycophenolate mofetil (MMF), and intravenous (IV) cyclophosphamide; (ii) oral corticosteroid (OCS); or (iii) a combination of (i) and (ii).
[A2.34] The pharmaceutical composition of A2.33, wherein the immunosuppressive agent comprises prednisone, prednisolone or equivalent thereof.
[A2.35] The pharmaceutical composition of any one of A2.1-A2.34, which improves the modified Rankin Scale (mRS) score improvement.
[A2.36] The pharmaceutical composition of A2.35, which
   reduces the time to the modified Rankin Scale (mRS) score improvement without use of rescue therapy
   delays the time to rescue therapy
   delays the time to seizure freedom or cessation of status epilepticus without use of rescue therapy
   improves the Clinical Assessment Scale of Encephalitis (CASE) score
   improves the Montreal Cognitive Assessment (MOCA) total score
   improves the Rey Auditory Verbal Learning Test (RAVLT) score for the subject with an anti-LGI1 antibody and/or
   improves the mRS score for the subject with an anti-NMDAR antibody.
[A2.37] The pharmaceutical composition of any one of A2.1-A2.36, which improves the subject's scores of one or more of the followings: modified Rankin Scale (mRS), Clinical Assessment Scale of Encephalitis (CASE), Montreal Cognitive Assessment (MOCA), Rey Auditory Verbal Learning Test (RAVLT), or Columbia-Suicide Severity Rating Scale (C-SSRS).
[A2.38] The pharmaceutical composition of any one of A2.1-A2.36, which reduces the subject's modified Rankin Scale (mRS) score, or Clinical Assessment Scale of Encephalitis (CASE) score; or increases the subject's Montreal Cognitive Assessment (MOCA) total score, or Rey Auditory Verbal Learning Test (RAVLT) score.
[A2.39] The pharmaceutical composition of any one of A2.1-A2.38, which is administered by a subcutaneous administration device.
[A2.40] The pharmaceutical composition of A2.39, wherein the the subcutaneous administration device is a pre-filled syringe (PFS) optionally comprising a needle safety device (PFS-NSD) or autoinjector (AI).
[B1] Use of an IL-6 inhibitor in the preparation of a medicament for treating or reducing risk of relapse of an autoimmune encephalitis (AIE) in a subject.
[B2] The use of B1, wherein the IL-6 inhibitor is an anti-IL-6 antibody or antigen-binding fragment thereof, or an anti-IL-6 receptor antibody or antigen binding fragment thereof.
[B3] The use of B1 or B2, wherein the IL-6 inhibitor is an anti-IL-6 receptor antibody or antigen binding fragment thereof.
[B4] The use of any one of B1-B3, wherein the IL-6 inhibitor is a humanized antibody.
[B5] The use of any one of B1-B4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.
[B6] The use of B5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.
[B7] The use of B5 or B6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.
[B8] The use of any one of B5-B7, wherein the IL-6 inhibitor is satralizumab.
[B9] The use of any one of B1-B8, wherein the medicament is for delaying relapse of, reducing frequency of relapse of, reducing severity of relapse of, or reducing the need for rescue therapy for relapse of the autoimmune encephalitis in the subject.
[B10] The use of any one of B1-B9, wherein the subject is positive for anti-N methyl-D aspartic acid receptor (NMDAR) antibody or anti-leucine rich glioma inactivated 1 (LGI1) antibody.
[B11] The use of any one of B1-B10, wherein the autoimmune encephalitis is a disease other than Bickerstaff's brainstem encephalitis, acute disseminated encephalomyelitis, Hashimoto's encephalopathy, primary angiitis of the central nervous system (CNS), and Rasmussen's encephalitis.
[B12] The use of any one of B1-B11, wherein an anti-NMDAR antibody is detected in the cerebral spinal fluid (CSF) from the subject using a cell-based assay.
[B13] The use of any one of B1-B12, wherein the autoimmune encephalitis is anti-NMDAR encephalitis.
[B14] The use of any one of B1-B11, wherein an anti-LGI1 antibody is detected in the serum or cerebral spinal fluid (CSF) from the subject using a cell-based assay.
[B15] The use of B14, wherein the subject has experienced working memory deficits, seizures, or psychiatric symptoms suggesting involvement of the limbic system.
[B16] The use of any one of B1-B11 and B14-B15, the autoimmune encephalitis is anti-LGI1 encephalitis.
[B17] The use of any one of B1-B16, wherein the autoimmune encephalitis is an anti-NMDAR or anti-LGI1 encephalitis.
[B18] The use of any one of B1-B17, wherein the subject is negative for anti-caspr2, anti-IgLON5, anti-DPPX, anti-GABA^{A}, anti-neurexin-3 alpha, and anti-myelin oligodendrocyte glycoprotein (MOG) antibodies.
[B19] The use of any one of B1-B18, wherein the subject is negative for cell surface neuronal antibodies or glial antibodies other than anti-NMDAR and anti-LGI1 antibodies.
[B20] The use of any one of B1-B19, wherein the subject is (i) anti-NMDAR antibody-positive and aged 12 years or older; or (ii) anti-LGI1 antibody-positive and aged 18 years or older.
[B21] The use of any one of B1-B20, wherein the subject is receiving no ongoing chronic immunosuppressive therapy.
[B22] The use of any one of B1-B20, wherein the subject is receiving ongoing treatment with a stable dose of azathioprine (AZA), mycophenolate mofetil (MMF), intravenous (IV) cyclophosphamide, oral corticosteroid (OCS), or a combination of AZA or MMF or IV cyclophosphamide and OCS.
[B23] The use of any one of B5-B22, wherein the medicament is characterized in that the medicament is used such that 60 mg or 120 mg, 120 mg or 180 mg, and 180 mg or 240 mg of the anti-IL-6 receptor antibody or antigen binding fragment thereof is administered to the subject with body weight of less than 40 kg, between 40 and 100 kg, and over 100 kg respectively for each administration.
[B24] The use of any one of B5-B22, wherein the medicament is characterized in that the medicament is used such that 60 mg of the anti-IL-6 receptor antibody or antigen binding fragment thereof is administered to the subject with body weight of less than 40 kg for each administration.
[B25] The use of any one of B5-B22, wherein the medicament is characterized in that the medicament is used such that 120 mg of the anti-IL-6 receptor antibody or antigen binding fragment thereof is administered to the subject with body weight of less than 40 kg for each administration.
[B26] The use of any one of B5-B22, wherein the medicament is characterized in that the medicament is used such that 120 mg of the anti-IL-6 receptor antibody or antigen binding fragment thereof is administered to the subject with body weight of between 40 and 100 kg for each administration.
[B27] The use of any one of B5-B22, wherein the medicament is characterized in that the medicament is used such that 180 mg of the anti-IL-6 receptor antibody or antigen binding fragment thereof is administered to the subject with body weight of between 40 and 100 kg for each administration.
[B28] The use of any one of B5-B22, wherein the medicament is characterized in that the medicament is used such that 180 mg of the anti-IL-6 receptor antibody or antigen binding fragment thereof is administered to the subject with body weight of over 100 kg for each administration.
[B29] The use of any one of B5-B22, wherein the medicament is characterized in that the medicament is used such that 240 mg of the anti-IL-6 receptor antibody or antigen binding fragment thereof is administered to the subject with body weight of over 100 kg for each administration.
[B30] The use of any one of B5-B29, wherein the medicament is characterized in that the medicament is used such that the anti-IL-6 receptor antibody or antigen binding fragment thereof is administered to the subject subcutaneously.
[B31] The use of any one of B5-B30, wherein the medicament is characterized in that the medicament is used such that the anti-IL-6 receptor antibody or antigen binding fragment thereof is administered to the subject every two weeks (Q2W) for three times, and thereafter every four weeks (Q4W).
[B32] The use of any one of B1-B31, wherein the medicament is characterized in that the medicament is used in combination with an immunosuppressive therapy (IST).
[B33] The use of B32, wherein the IST is a therapy with (i) an immunosuppressive agent selected from the group consisting of azathioprine (AZA), mycophenolate mofetil (MMF), and intravenous (IV) cyclophosphamide; (ii) oral corticosteroid (OCS); or (iii) a combination of (i) and (ii).
[B34] The use of B33, wherein the immunosuppressive agent comprises prednisone, prednisolone or equivalent thereof.
[B35] The use of any one of B1-B34, wherein the medicament improves the modified Rankin Scale (mRS) score improvement.
[B36] The use of B35, wherein the medicament
   reduces the time to the modified Rankin Scale (mRS) score improvement without use of rescue therapy
   delays the time to rescue therapy
   delays the time to seizure freedom or cessation of status epilepticus without use of rescue therapy
   improves the Clinical Assessment Scale of Encephalitis (CASE) score
   improves the Montreal Cognitive Assessment (MOCA) total score
   improves the Rey Auditory Verbal Learning Test (RAVLT) score for the subject with an anti-LGI1 antibody and/or
   improves the mRS score for the subject with an anti-NMDAR antibody.
[B37] The use of any one of B1-B36, wherein the medicament improves the subject's scores of one or more of the followings: modified Rankin Scale (mRS), Clinical Assessment Scale of Encephalitis (CASE), Montreal Cognitive Assessment (MOCA), Rey Auditory Verbal Learning Test (RAVLT), or Columbia-Suicide Severity Rating Scale (C-SSRS).
[B38] The use of any one of B1-B36, wherein the medicament reduces the subject's modified Rankin Scale (mRS) score, or Clinical Assessment Scale of Encephalitis (CASE) score; or increases the subject's Montreal Cognitive Assessment (MOCA) total score, or Rey Auditory Verbal Learning Test (RAVLT) score.
[B39] The use of any one of B1-B38, wherein the medicament is administered by a subcutaneous administration device.
[B40] The use of B39, wherein the the subcutaneous administration device is a pre-filled syringe (PFS) optionally comprising a needle safety device (PFS-NSD) or autoinjector (AI).
[C1] An IL-6 inhibitor for use in treating or reducing risk of relapse of an autoimmune encephalitis (AIE) in a subject.
[C2] The IL-6 inhibitor for use of C1, wherein the IL-6 inhibitor is an anti-IL-6 antibody or antigen-binding fragment thereof, or an anti-IL-6 receptor antibody or antigen binding fragment thereof.
[C3] The IL-6 inhibitor for use of C1 or C2, wherein the IL-6 inhibitor is an anti-IL-6 receptor antibody or antigen binding fragment thereof.
[C4] The IL-6 inhibitor for use of any one of C1-C3, wherein the IL-6 inhibitor is a humanized antibody.
[C5] The IL-6 inhibitor for use of any one of C1-C4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.
[C6] The IL-6 inhibitor for use of C5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.
[C7] The IL-6 inhibitor for use of C5 or C6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.
[C8] The IL-6 inhibitor for use of any one of C5-C7, wherein the IL-6 inhibitor is satralizumab.
[C9] The IL-6 inhibitor for use of any one of C1-C8, for delaying relapse of, reducing frequency of relapse of, reducing severity of relapse of, or reducing the need for rescue therapy for relapse of the autoimmune encephalitis in the subject.
[C10] The IL-6 inhibitor for use of any one of C1-C9, wherein the subject is positive for anti-N methyl-D aspartic acid receptor (NMDAR) antibody or anti-leucine rich glioma inactivated 1 (LGI1) antibody.
[C11] The IL-6 inhibitor for use of any one of C1-C10, wherein the autoimmune encephalitis is a disease other than Bickerstaff's brainstem encephalitis, acute disseminated encephalomyelitis, Hashimoto's encephalopathy, primary angiitis of the central nervous system (CNS), and Rasmussen's encephalitis.
[C12] The IL-6 inhibitor for use of any one of C1-C11, wherein an anti-NMDAR antibody is detected in the cerebral spinal fluid (CSF) from the subject using a cell-based assay.
[C13] The IL-6 inhibitor for use of any one of C1-C12, wherein the autoimmune encephalitis is anti-NMDAR encephalitis.
[C14] The IL-6 inhibitor for use of any one of C1-C11, wherein an anti-LGI1 antibody is detected in the serum or cerebral spinal fluid (CSF) from the subject using a cell-based assay.
[C15] The IL-6 inhibitor for use of C14, wherein the subject has experienced working memory deficits, seizures, or psychiatric symptoms suggesting involvement of the limbic system.
[C16] The IL-6 inhibitor for use of any one of C1-C11 and C14-C15, the autoimmune encephalitis is anti-LGI1 encephalitis.
[C17] The IL-6 inhibitor for use of any one of C1-C16, wherein the autoimmune encephalitis is an anti-NMDAR or anti-LGI1 encephalitis.
[C18] The IL-6 inhibitor for use of any one of C1-C17, wherein the subject is negative for anti-caspr2, anti-IgLON5, anti-DPPX, anti-GABA^{A}, anti-neurexin-3 alpha, and anti-myelin oligodendrocyte glycoprotein (MOG) antibodies.
[C19] The IL-6 inhibitor for use of any one of C1-C18, wherein the subject is negative for cell surface neuronal antibodies or glial antibodies other than anti-NMDAR and anti-LGI1 antibodies.
[C20] The IL-6 inhibitor for use of any one of C1-C19, wherein the subject is (i) anti-NMDAR antibody-positive and aged 12 years or older; or (ii) anti-LGI1 antibody-positive and aged 18 years or older.
[C21] The IL-6 inhibitor for use of any one of C1-C20, wherein the subject is receiving no ongoing chronic immunosuppressive therapy.
[C22] The IL-6 inhibitor for use of any one of C1-C20, wherein the subject is receiving ongoing treatment with a stable dose of azathioprine (AZA), mycophenolate mofetil (MMF), intravenous (IV) cyclophosphamide, oral corticosteroid (OCS), or a combination of AZA or MMF or IV cyclophosphamide and OCS.
[C23] The IL-6 inhibitor for use of any one of C5-C22, which is characterized in that 60 mg or 120 mg, 120 mg or 180 mg, and 180 mg or 240 mg of the anti-IL-6 receptor antibody or antigen binding fragment thereof is administered to the subject with body weight of less than 40 kg, between 40 and 100 kg, and over 100 kg respectively for each administration.
[C24] The IL-6 inhibitor for use of any one of C5-C22, which is characterized in that 60 mg of the anti-IL-6 receptor antibody or antigen binding fragment thereof is administered to the subject with body weight of less than 40 kg for each administration.
[C25] The IL-6 inhibitor for use of any one of C5-C22, which is characterized in that 120 mg of the anti-IL-6 receptor antibody or antigen binding fragment thereof is administered to the subject with body weight of less than 40 kg for each administration.
[C26] The IL-6 inhibitor for use of any one of C5-C22, which is characterized in that 120 mg of the anti-TL-6 receptor antibody or antigen binding fragment thereof is administered to the subject with body weight of between 40 and 100 kg for each administration.
[C27] The IL-6 inhibitor for use of any one of C5-C22, which is characterized in that 180 mg of the anti-IL-6 receptor antibody or antigen binding fragment thereof is administered to the subject with body weight of between 40 and 100 kg for each administration.
[C28] The IL-6 inhibitor for use of any one of C5-C22, which is characterized in that 180 mg of the anti-IL-6 receptor antibody or antigen binding fragment thereof is administered to the subject with body weight of over 100 kg for each administration.
[C29] The IL-6 inhibitor for use of any one of C5-C22, which is characterized in that 240 mg of the anti-IL-6 receptor antibody or antigen binding fragment thereof is administered to the subject with body weight of over 100 kg for each administration.
[C30] The IL-6 inhibitor for use of any one of C5-C29, which is characterized in that the anti-IL-6 receptor antibody or antigen binding fragment thereof is administered to the subject subcutaneously.
[C31] The IL-6 inhibitor for use of any one of C5-C30, which is characterized in that the anti-IL-6 receptor antibody or antigen binding fragment thereof is administered to the subject every two weeks (Q2W) for three times, and thereafter every four weeks (Q4W).
[C32] The IL-6 inhibitor for use of any one of C1-C31, which is used in combination with an immunosuppressive therapy (IST).
[C33] The IL-6 inhibitor for use of C32, wherein the IST is a therapy with (i) an immunosuppressive agent selected from the group consisting of azathioprine (AZA), mycophenolate mofetil (MMF), and intravenous (IV) cyclophosphamide; (ii) oral corticosteroid (OCS); or (iii) a combination of (i) and (ii).
[C34] The IL-6 inhibitor for use of C33, wherein the immunosuppressive agent comprises prednisone, prednisolone or equivalent thereof.
[C35] The IL-6 inhibitor for use of any one of C1-C34, which improves the modified Rankin Scale (mRS) score improvement.
[C36] The IL-6 inhibitor for use of C35, which
   reduces the time to the modified Rankin Scale (mRS) score improvement without use of rescue therapy
   delays the time to rescue therapy
   delays the time to seizure freedom or cessation of status epilepticus without use of rescue therapy
   improves the Clinical Assessment Scale of Encephalitis (CASE) score
   improves the Montreal Cognitive Assessment (MOCA) total score
   improves the Rey Auditory Verbal Learning Test (RAVLT) score for the subject with an anti-LGI1 antibody and/or
   improves the mRS score for the subject with an anti-NMDAR antibody.
[C37] The IL-6 inhibitor for use of any one of C1-C36, which improves the subject's scores of one or more of the followings: modified Rankin Scale (mRS), Clinical Assessment Scale of Encephalitis (CASE), Montreal Cognitive Assessment (MOCA), Rey Auditory Verbal Learning Test (RAVLT), or Columbia-Suicide Severity Rating Scale (C-SSRS).
[C38] The IL-6 inhibitor for use of any one of C1-C36, which reduces the subject's modified Rankin Scale (mRS) score, or Clinical Assessment Scale of Encephalitis (CASE) score; or increases the subject's Montreal Cognitive Assessment (MOCA) total score, or Rey Auditory Verbal Learning Test (RAVLT) score.
[C39] The IL-6 inhibitor for use of any one of C1-C38, which is administered by a subcutaneous administration device.
[C40] The IL-6 inhibitor for use of C39, wherein the the subcutaneous administration device is a pre-filled syringe (PFS) optionally comprising a needle safety device (PFS-NSD) or autoinjector (AI).
[C2.1] Satralizumab for use in treating anti-N methyl-D aspartic acid receptor (NMDAR) encephalitis.
[C2.2] Satralizumab for use in treating anti-N methyl-D aspartic acid receptor (NMDAR) encephalitis; wherein 60 mg of satralizumab for each administration is administered subcutaneously to the subject with body weight of less than 40 kg every two weeks (Q2W) for three times, and thereafter every four weeks (Q4W).
[C2.3] Satralizumab for use in treating anti-N methyl-D aspartic acid receptor (NMDAR) encephalitis; wherein 120 mg of satralizumab for each administration is administered subcutaneously to the subject with body weight of less than 40 kg every two weeks (Q2W) for three times, and thereafter every four weeks (Q4W).
[C2.4] Satralizumab for use in treating anti-N methyl-D aspartic acid receptor (NMDAR) encephalitis; wherein 120 mg of satralizumab for each administration is administered subcutaneously to the subject with body weight of between 40 and 100 kg every two weeks (Q2W) for three times, and thereafter every four weeks (Q4W).
[C2.5] Satralizumab for use in treating anti-N methyl-D aspartic acid receptor (NMDAR) encephalitis; wherein 180 mg of satralizumab for each administration is administered subcutaneously to the subject with body weight of between 40 and 100 kg every two weeks (Q2W) for three times, and thereafter every four weeks (Q4W).
[C2.6] Satralizumab for use in treating anti-N methyl-D aspartic acid receptor (NMDAR) encephalitis; wherein 180 mg of satralizumab for each administration is administered subcutaneously to the subject with body weight of over 100 kg every two weeks (Q2W) for three times, and thereafter every four weeks (Q4W).
[C2.7] Satralizumab for use in treating anti-N methyl-D aspartic acid receptor (NMDAR) encephalitis; wherein 240 mg of satralizumab for each administration is administered subcutaneously to the subject with body weight of over 100 kg every two weeks (Q2W) for three times, and thereafter every four weeks (Q4W).
[C2.8] Satralizumab for use in treating anti-N methyl-D aspartic acid receptor (NMDAR) encephalitis; wherein satralizumab is used in combination with azathioprine (AZA) or mycophenolate mofetil (MMF) or intravenous (IV) cyclophosphamide.
[C2.9] Satralizumab for use in treating anti-N methyl-D aspartic acid receptor (NMDAR) encephalitis; wherein 60 mg of satralizumab for each administration is administered subcutaneously to the subject with body weight of less than 40 kg every two weeks (Q2W) for three times, and thereafter every four weeks (Q4W); and wherein satralizumab is used in combination with azathioprine (AZA) or mycophenolate mofetil (MMF) or intravenous (IV) cyclophosphamide.
[C2.10] Satralizumab for use in treating anti-N methyl-D aspartic acid receptor (NMDAR) encephalitis; wherein 120 mg of satralizumab for each administration is administered subcutaneously to the subject with body weight of less than 40 kg every two weeks (Q2W) for three times, and thereafter every four weeks (Q4W); and wherein satralizumab is used in combination with azathioprine (AZA) or mycophenolate mofetil (MMF) or intravenous (IV) cyclophosphamide.
[C2.11] Satralizumab for use in treating anti-N methyl-D aspartic acid receptor (NMDAR) encephalitis; wherein 120 mg of satralizumab for each administration is administered subcutaneously to the subject with body weight of between 40 and 100 kg every two weeks (Q2W) for three times, and thereafter every four weeks (Q4W); and wherein satralizumab is used in combination with azathioprine (AZA) or mycophenolate mofetil (MMF) or intravenous (IV) cyclophosphamide.
[C2.12] Satralizumab for use in treating anti-N methyl-D aspartic acid receptor (NMDAR) encephalitis; wherein 180 mg of satralizumab for each administration is administered subcutaneously to the subject with body weight of between 40 and 100 kg every two weeks (Q2W) for three times, and thereafter every four weeks (Q4W); and wherein satralizumab is used in combination with azathioprine (AZA) or mycophenolate mofetil (MMF) or intravenous (IV) cyclophosphamide.
[C2.13] Satralizumab for use in treating anti-N methyl-D aspartic acid receptor (NMDAR) encephalitis; wherein 180 mg of satralizumab for each administration is administered subcutaneously to the subject with body weight of over 100 kg every two weeks (Q2W) for three times, and thereafter every four weeks (Q4W); and wherein satralizumab is used in combination with azathioprine (AZA) or mycophenolate mofetil (MMF) or intravenous (IV) cyclophosphamide.
[C2.14] Satralizumab for use in treating anti-N methyl-D aspartic acid receptor (NMDAR) encephalitis; wherein 240 mg of satralizumab for each administration is administered subcutaneously to the subject with body weight of over 100 kg every two weeks (Q2W) for three times, and thereafter every four weeks (Q4W); and wherein satralizumab is used in combination with azathioprine (AZA) or mycophenolate mofetil (MMF) or intravenous (IV) cyclophosphamide.
[C2.15] Satralizumab for use in treating anti-N methyl-D aspartic acid receptor (NMDAR) encephalitis; wherein satralizumab is used in combination with oral corticosteroid (OCS).
[C2.16] Satralizumab for use in treating anti-N methyl-D aspartic acid receptor (NMDAR) encephalitis; wherein 60 mg of satralizumab for each administration is administered subcutaneously to the subject with body weight of less than 40 kg every two weeks (Q2W) for three times, and thereafter every four weeks (Q4W); and wherein satralizumab is used in combination with oral corticosteroid (OCS).
[C2.17] Satralizumab for use in treating anti-N methyl-D aspartic acid receptor (NMDAR) encephalitis; wherein 120 mg of satralizumab for each administration is administered subcutaneously to the subject with body weight of less than 40 kg every two weeks (Q2W) for three times, and thereafter every four weeks (Q4W); and wherein satralizumab is used in combination with oral corticosteroid (OCS).
[C2.18] Satralizumab for use in treating anti-N methyl-D aspartic acid receptor (NMDAR) encephalitis; wherein 120 mg of satralizumab for each administration is administered subcutaneously to the subject with body weight of between 40 and 100 kg every two weeks (Q2W) for three times, and thereafter every four weeks (Q4W); and wherein satralizumab is used in combination with oral corticosteroid (OCS).
[C2.19] Satralizumab for use in treating anti-N methyl-D aspartic acid receptor (NMDAR) encephalitis; wherein 180 mg of satralizumab for each administration is administered subcutaneously to the subject with body weight of between 40 and 100 kg every two weeks (Q2W) for three times, and thereafter every four weeks (Q4W); and wherein satralizumab is used in combination with oral corticosteroid (OCS).
[C2.20] Satralizumab for use in treating anti-N methyl-D aspartic acid receptor (NMDAR) encephalitis; wherein 180 mg of satralizumab for each administration is administered subcutaneously to the subject with body weight of over 100 kg every two weeks (Q2W) for three times, and thereafter every four weeks (Q4W); and wherein satralizumab is used in combination with oral corticosteroid (OCS).
[C2.21] Satralizumab for use in treating anti-N methyl-D aspartic acid receptor (NMDAR) encephalitis; wherein 240 mg of satralizumab for each administration is administered subcutaneously to the subject with body weight of over 100 kg every two weeks (Q2W) for three times, and thereafter every four weeks (Q4W); and wherein satralizumab is used in combination with oral corticosteroid (OCS).
[C2.22] Satralizumab for use in treating anti-N methyl-D aspartic acid receptor (NMDAR) encephalitis; wherein satralizumab is used in combination with (i) any one of azathioprine (AZA), mycophenolate mofetil (MMF) and intravenous (IV) cyclophosphamide, and (ii) oral corticosteroid (OCS).
[C2.23] Satralizumab for use in treating anti-N methyl-D aspartic acid receptor (NMDAR) encephalitis; wherein 60 mg of satralizumab for each administration is administered subcutaneously to the subject with body weight of less than 40 kg every two weeks (Q2W) for three times, and thereafter every four weeks (Q4W); and wherein satralizumab is used in combination with (i) any one of azathioprine (AZA), mycophenolate mofetil (MMF) and intravenous (IV) cyclophosphamide, and (ii) oral corticosteroid (OCS).
[C2.24] Satralizumab for use in treating anti-N methyl-D aspartic acid receptor (NMDAR) encephalitis; wherein 120 mg of satralizumab for each administration is administered subcutaneously to the subject with body weight of less than 40 kg every two weeks (Q2W) for three times, and thereafter every four weeks (Q4W); and wherein satralizumab is used in combination with (i) any one of azathioprine (AZA), mycophenolate mofetil (MMF) and intravenous (IV) cyclophosphamide, and (ii) oral corticosteroid (OCS).
[C2.25] Satralizumab for use in treating anti-N methyl-D aspartic acid receptor (NMDAR) encephalitis; wherein 120 mg of satralizumab for each administration is administered subcutaneously to the subject with body weight of between 40 and 100 kg every two weeks (Q2W) for three times, and thereafter every four weeks (Q4W); and wherein satralizumab is used in combination with (i) any one of azathioprine (AZA), mycophenolate mofetil (MMF) and intravenous (IV) cyclophosphamide, and (ii) oral corticosteroid (OCS).
[C2.26] Satralizumab for use in treating anti-N methyl-D aspartic acid receptor (NMDAR) encephalitis; wherein 180 mg of satralizumab for each administration is administered subcutaneously to the subject with body weight of between 40 and 100 kg every two weeks (Q2W) for three times, and thereafter every four weeks (Q4W); and wherein satralizumab is used in combination with (i) any one of azathioprine (AZA), mycophenolate mofetil (MMF) and intravenous (IV) cyclophosphamide, and (ii) oral corticosteroid (OCS).
[C2.27] Satralizumab for use in treating anti-N methyl-D aspartic acid receptor (NMDAR) encephalitis; wherein 180 mg of satralizumab for each administration is administered subcutaneously to the subject with body weight of over 100 kg every two weeks (Q2W) for three times, and thereafter every four weeks (Q4W); and wherein satralizumab is used in combination with (i) any one of azathioprine (AZA), mycophenolate mofetil (MMF) and intravenous (IV) cyclophosphamide, and (ii) oral corticosteroid (OCS).
[C2.28] Satralizumab for use in treating anti-N methyl-D aspartic acid receptor (NMDAR) encephalitis; wherein 240 mg of satralizumab for each administration is administered subcutaneously to the subject with body weight of over 100 kg every two weeks (Q2W) for three times, and thereafter every four weeks (Q4W); and wherein satralizumab is used in combination with (i) any one of azathioprine (AZA), mycophenolate mofetil (MMF) and intravenous (IV) cyclophosphamide, and (ii) oral corticosteroid (OCS).
[C2.29] Satralizumab for use in treating anti-leucine rich glioma inactivated 1 (LGI1) encephalitis.
[C2.30] Satralizumab for use in treating anti-leucine rich glioma inactivated 1 (LGI1) encephalitis; wherein 60 mg of satralizumab for each administration is administered subcutaneously to the subject with body weight of less than 40 kg every two weeks (Q2W) for three times, and thereafter every four weeks (Q4W).
[C2.31] Satralizumab for use in treating anti-leucine rich glioma inactivated 1 (LGI1) encephalitis; wherein 120 mg of satralizumab for each administration is administered subcutaneously to the subject with body weight of less than 40 kg every two weeks (Q2W) for three times, and thereafter every four weeks (Q4W).
[C2.32] Satralizumab for use in treating anti-leucine rich glioma inactivated 1 (LGI1) encephalitis; wherein 120 mg of satralizumab for each administration is administered subcutaneously to the subject with body weight of between 40 and 100 kg every two weeks (Q2W) for three times, and thereafter every four weeks (Q4W).
[C2.33] Satralizumab for use in treating anti-leucine rich glioma inactivated 1 (LGI1) encephalitis; wherein 180 mg of satralizumab for each administration is administered subcutaneously to the subject with body weight of between 40 and 100 kg every two weeks (Q2W) for three times, and thereafter every four weeks (Q4W).
[C2.34] Satralizumab for use in treating anti-leucine rich glioma inactivated 1 (LGI1) encephalitis; wherein 180 mg of satralizumab for each administration is administered subcutaneously to the subject with body weight of over 100 kg every two weeks (Q2W) for three times, and thereafter every four weeks (Q4W).
[C2.35] Satralizumab for use in treating anti-leucine rich glioma inactivated 1 (LGI1) encephalitis; wherein 240 mg of satralizumab for each administration is administered subcutaneously to the subject with body weight of over 100 kg every two weeks (Q2W) for three times, and thereafter every four weeks (Q4W).
[C2.36] Satralizumab for use in treating anti-leucine rich glioma inactivated 1 (LGI1) encephalitis; wherein satralizumab is used in combination with azathioprine (AZA) or mycophenolate mofetil (MMF) or intravenous (IV) cyclophosphamide.
[C2.37] Satralizumab for use in treating anti-leucine rich glioma inactivated 1 (LGI1) encephalitis; wherein 60 mg of satralizumab for each administration is administered subcutaneously to the subject with body weight of less than 40 kg every two weeks (Q2W) for three times, and thereafter every four weeks (Q4W); and wherein satralizumab is used in combination with azathioprine (AZA) or mycophenolate mofetil (MMF) or intravenous (IV) cyclophosphamide.
[C2.38] Satralizumab for use in treating anti-leucine rich glioma inactivated 1 (LGI1) encephalitis; wherein 120 mg of satralizumab for each administration is administered subcutaneously to the subject with body weight of less than 40 kg every two weeks (Q2W) for three times, and thereafter every four weeks (Q4W); and wherein satralizumab is used in combination with azathioprine (AZA) or mycophenolate mofetil (MMF) or intravenous (IV) cyclophosphamide.
[C2.39] Satralizumab for use in treating anti-leucine rich glioma inactivated 1 (LGI1) encephalitis; wherein 120 mg of satralizumab for each administration is administered subcutaneously to the subject with body weight of between 40 and 100 kg every two weeks (Q2W) for three times, and thereafter every four weeks (Q4W); and wherein satralizumab is used in combination with azathioprine (AZA) or mycophenolate mofetil (MMF) or intravenous (IV) cyclophosphamide.
[C2.40] Satralizumab for use in treating anti-leucine rich glioma inactivated 1 (LGI1) encephalitis; wherein 180 mg of satralizumab for each administration is administered subcutaneously to the subject with body weight of between 40 and 100 kg every two weeks (Q2W) for three times, and thereafter every four weeks (Q4W); and wherein satralizumab is used in combination with azathioprine (AZA) or mycophenolate mofetil (MMF) or intravenous (IV) cyclophosphamide.
[C2.41] Satralizumab for use in treating anti-leucine rich glioma inactivated 1 (LGI1) encephalitis; wherein 180 mg of satralizumab for each administration is administered subcutaneously to the subject with body weight of over 100 kg every two weeks (Q2W) for three times, and thereafter every four weeks (Q4W); and wherein satralizumab is used in combination with azathioprine (AZA) or mycophenolate mofetil (MMF) or intravenous (IV) cyclophosphamide.
[C2.42] Satralizumab for use in treating anti-leucine rich glioma inactivated 1 (LGI1) encephalitis; wherein 240 mg of satralizumab for each administration is administered subcutaneously to the subject with body weight of over 100 kg every two weeks (Q2W) for three times, and thereafter every four weeks (Q4W); and wherein satralizumab is used in combination with azathioprine (AZA) or mycophenolate mofetil (MMF) or intravenous (IV) cyclophosphamide.
[C2.43] Satralizumab for use in treating anti-leucine rich glioma inactivated 1 (LGI1) encephalitis; wherein satralizumab is used in combination with oral corticosteroid (OCS).
[C2.44] Satralizumab for use in treating anti-leucine rich glioma inactivated 1 (LGI1) encephalitis; wherein 60 mg of satralizumab for each administration is administered subcutaneously to the subject with body weight of less than 40 kg every two weeks (Q2W) for three times, and thereafter every four weeks (Q4W); and wherein satralizumab is used in combination with oral corticosteroid (OCS).
[C2.45] Satralizumab for use in treating anti-leucine rich glioma inactivated 1 (LGI1) encephalitis; wherein 120 mg of satralizumab for each administration is administered subcutaneously to the subject with body weight of less than 40 kg every two weeks (Q2W) for three times, and thereafter every four weeks (Q4W); and wherein satralizumab is used in combination with oral corticosteroid (OCS).
[C2.46] Satralizumab for use in treating anti-leucine rich glioma inactivated 1 (LGI1) encephalitis; wherein 120 mg of satralizumab for each administration is administered subcutaneously to the subject with body weight of between 40 and 100 kg every two weeks (Q2W) for three times, and thereafter every four weeks (Q4W); and wherein satralizumab is used in combination with oral corticosteroid (OCS).
[C2.47] Satralizumab for use in treating anti-leucine rich glioma inactivated 1 (LGI1) encephalitis; wherein 180 mg of satralizumab for each administration is administered subcutaneously to the subject with body weight of between 40 and 100 kg every two weeks (Q2W) for three times, and thereafter every four weeks (Q4W); and wherein satralizumab is used in combination with oral corticosteroid (OCS).
[C2.48] Satralizumab for use in treating anti-leucine rich glioma inactivated 1 (LGI1) encephalitis; wherein 180 mg of satralizumab for each administration is administered subcutaneously to the subject with body weight of over 100 kg every two weeks (Q2W) for three times, and thereafter every four weeks (Q4W); and wherein satralizumab is used in combination with oral corticosteroid (OCS).
[C2.49] Satralizumab for use in treating anti-leucine rich glioma inactivated 1 (LGI1) encephalitis; wherein 240 mg of satralizumab for each administration is administered subcutaneously to the subject with body weight of over 100 kg every two weeks (Q2W) for three times, and thereafter every four weeks (Q4W); and wherein satralizumab is used in combination with oral corticosteroid (OCS).
[C2.50] Satralizumab for use in treating anti-leucine rich glioma inactivated 1 (LGI1) encephalitis; wherein satralizumab is used in combination with (i) any one of azathioprine (AZA), mycophenolate mofetil (MMF) and intravenous (IV) cyclophosphamide, and (ii) oral corticosteroid (OCS).
[C2.51] Satralizumab for use in treating anti-leucine rich glioma inactivated 1 (LGI1) encephalitis; wherein 60 mg of satralizumab for each administration is administered subcutaneously to the subject with body weight of less than 40 kg every two weeks (Q2W) for three times, and thereafter every four weeks (Q4W); and wherein satralizumab is used in combination with (i) any one of azathioprine (AZA), mycophenolate mofetil (MMF) and intravenous (IV) cyclophosphamide, and (ii) oral corticosteroid (OCS).
[C2.52] Satralizumab for use in treating anti-leucine rich glioma inactivated 1 (LGI1) encephalitis; wherein 120 mg of satralizumab for each administration is administered subcutaneously to the subject with body weight of less than 40 kg every two weeks (Q2W) for three times, and thereafter every four weeks (Q4W); and wherein satralizumab is used in combination with (i) any one of azathioprine (AZA), mycophenolate mofetil (MMF) and intravenous (IV) cyclophosphamide, and (ii) oral corticosteroid (OCS).
[C2.53] Satralizumab for use in treating anti-leucine rich glioma inactivated 1 (LGI1) encephalitis; wherein 120 mg of satralizumab for each administration is administered subcutaneously to the subject with body weight of between 40 and 100 kg every two weeks (Q2W) for three times, and thereafter every four weeks (Q4W); and wherein satralizumab is used in combination with (i) any one of azathioprine (AZA), mycophenolate mofetil (MMF) and intravenous (IV) cyclophosphamide, and (ii) oral corticosteroid (OCS).
[C2.54] Satralizumab for use in treating anti-leucine rich glioma inactivated 1 (LGI1) encephalitis; wherein 180 mg of satralizumab for each administration is administered subcutaneously to the subject with body weight of between 40 and 100 kg every two weeks (Q2W) for three times, and thereafter every four weeks (Q4W); and wherein satralizumab is used in combination with (i) any one of azathioprine (AZA), mycophenolate mofetil (MMF) and intravenous (IV) cyclophosphamide, and (ii) oral corticosteroid (OCS).
[C2.55] Satralizumab for use in treating anti-leucine rich glioma inactivated 1 (LGI1) encephalitis; wherein 180 mg of satralizumab for each administration is administered subcutaneously to the subject with body weight of over 100 kg every two weeks (Q2W) for three times, and thereafter every four weeks (Q4W); and wherein satralizumab is used in combination with (i) any one of azathioprine (AZA), mycophenolate mofetil (MMF) and intravenous (IV) cyclophosphamide, and (ii) oral corticosteroid (OCS).
[C2.56] Satralizumab for use in treating anti-leucine rich glioma inactivated 1 (LGI1) encephalitis; wherein 240 mg of satralizumab for each administration is administered subcutaneously to the subject with body weight of over 100 kg every two weeks (Q2W) for three times, and thereafter every four weeks (Q4W); and wherein satralizumab is used in combination with (i) any one of azathioprine (AZA), mycophenolate mofetil (MMF) and intravenous (IV) cyclophosphamide, and (ii) oral corticosteroid (OCS).
[D1] A kit for treating or reducing risk of relapse of an autoimmune encephalitis (AIE) in a subject, comprising:
(1) the pharmaceutical composition of any one of A2.1-A2.40; and
(2) a package insert or label instructing administration of the pharmaceutical composition to a subject.
[D2] A subcutaneous administration device is a pre-filled syringe (PFS) optionally comprising a needle safety device (PFS-NSD) or autoinjector (AI).
[D2] A subcutaneous administration device comprising a fixed dose of 60 mg of satralizumab in a pharmaceutically acceptable excipient.
[D3] A subcutaneous administration device comprising a fixed dose of 240 mg of satralizumab in a pharmaceutically acceptable excipient.
[D4] The subcutaneous administration device of D2 or D3, wherein the device is a prefilled syringe (PFS) optionally comprising a needle safety device (PFS-NSD).
[D5] The subcutaneous administration device of D2 or D3, wherein the device is an autoinjector (AI).
[D6] A subcutaneous administration device comprising a fixed dose of 60 mg of satralizumab in a pharmaceutically acceptable excipient, wherein the device is a prefilled syringe comprising a needle safety device (PFS-NSD).
[D7] A subcutaneous administration device comprising a fixed dose of 120 mg of satralizumab in a pharmaceutically acceptable excipient, wherein the device is a prefilled syringe comprising a needle safety device (PFS-NSD).
[D8] A subcutaneous administration device comprising a fixed dose of 120 mg, 180 mg, or 240 mg of satralizumab in a pharmaceutically acceptable excipient, wherein the device is an autoinjector (AI).
[E1] A method of treating a subject having an autoimmune encephalitis (AIE), the method comprising administering to the subject an effective amount of an IL-6 inhibitor.
[E2] The method of E1, wherein the IL-6 inhibitor is an anti-IL-6 antibody or antigen-binding fragment thereof, or an anti-IL-6 receptor antibody or antigen binding fragment thereof.
[E3] The method of E1 or E2, wherein the IL-6 inhibitor is an anti-IL-6 receptor antibody or antigen binding fragment thereof.
[E4] The method of any one of E1-E3, wherein the IL-6 inhibitor is a humanized antibody.
[E5] The method of any one of E1-E4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.
[E6] The method of E5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.
[E7] The method of E5 or E6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.
[E8] The method of any one of E5-E7, wherein the IL-6 inhibitor is satralizumab.
[E9] The method of any one of E1-E8, wherein the subject is positive for anti-N methyl-D aspartic acid receptor (NMDAR) antibody or anti-leucine rich glioma inactivated 1 (LGI1) antibody.
[E10] The method of any one of E1-E9, wherein the autoimmune encephalitis is a disease other than Bickerstaff's brainstem encephalitis, acute disseminated encephalomyelitis, Hashimoto's encephalopathy, primary angiitis of the central nervous system (CNS), and Rasmussen's encephalitis.
[E11] The method of any one of E1-E10, wherein an anti-NMDAR antibody is detected in the cerebral spinal fluid (CSF) from the subject using a cell-based assay.
[E12] The method of any one of E1-E11, wherein the autoimmune encephalitis is anti-NMDAR encephalitis.
[E13] The method of any one of E1-E10, wherein an anti-LGI1 antibody is detected in the serum or cerebral spinal fluid (CSF) from the subject using a cell-based assay.
[E14] The method of E13, wherein the subject has experienced working memory deficits, seizures, or psychiatric symptoms suggesting involvement of the limbic system.
[E15] The method of any one of E1-E10 and E13-E14, the autoimmune encephalitis is anti-LGI1 encephalitis.
[E16] The method of any one of E1-E15, wherein the autoimmune encephalitis is an anti-NMDAR or anti-LGI1 encephalitis.
[E17] The method of any one of E1-E16, wherein the subject is negative for anti-caspr2, anti-IgLON5, anti-DPPX, anti-GABA^{A}, anti-neurexin-3 alpha, and anti-myelin oligodendrocyte glycoprotein (MOG) antibodies.
[E18] The method of any one of E1-E17, wherein the subject is negative for cell surface neuronal antibodies or glial antibodies other than anti-NMDAR and anti-LGI1 antibodies.
[E19] The method of any one of E1-E18, wherein the subject is (i) anti-NMDAR antibody-positive and aged 12 years or older; or (ii) anti-LGI1 antibody-positive and aged 18 years or older.
[E20] The method of any one of E1-E19, wherein the subject is receiving no ongoing chronic immunosuppressive therapy.
[E21] The method of any one of E1-E19, wherein the subject is receiving ongoing treatment with a stable dose of azathioprine (AZA), mycophenolate mofetil (MMF), intravenous (IV) cyclophosphamide, oral corticosteroid (OCS), or a combination of AZA or MMF or IV cyclophosphamide and OCS.
[E22] The method of any one of E5-E21, wherein
the subject is determined to have a body weight of less than 40 kg, and the amount of the anti-IL-6 receptor antibody or antigen binding fragment thereof administered to the subject in each administration is 60 mg or 120 mg; or
the subject is determined to have a body weight of between 40 and 100 kg, and the amount of the anti-IL-6 receptor antibody or antigen binding fragment thereof administered to the subject in each administration is 120 mg or 180 mg; or
the subject is determined to have a body weight of over 100 kg, and the amount of the anti-IL-6 receptor antibody or antigen binding fragment thereof administered to the subject in each administration is 180 mg or 240 mg.
[E23] The method of any one of E5-E21, wherein the subject is determined to have a body weight of less than 40 kg, and the amount of the anti-IL-6 receptor antibody or antigen binding fragment thereof administered to the subject in each administration is 60 mg.
[E24] The method of any one of E5-E21, wherein the subject is determined to have a body weight of less than 40 kg, and the amount of the anti-IL-6 receptor antibody or antigen binding fragment thereof administered to the subject in each administration is 120 mg.
[E25] The method of any one of E5-E21, wherein the subject is determined to have a body weight of between 40 and 100 kg, and the amount of the anti-IL-6 receptor antibody or antigen binding fragment thereof administered to the subject in each administration is 120 mg.
[E26] The method of any one of E5-E21, wherein the subject is determined to have a body weight of between 40 and 100 kg, and the amount of the anti-IL-6 receptor antibody or antigen binding fragment thereof administered to the subject in each administration is 180 mg.
[E27] The method of any one of E5-E21, wherein the subject is determined to have a body weight of over 100 kg, and the amount of the anti-IL-6 receptor antibody or antigen binding fragment thereof administered to the subject in each administration is 180 mg.
[E28] The method of any one of E5-E21, wherein the subject is determined to have a body weight of over 100 kg, and the amount of the anti-IL-6 receptor antibody or antigen binding fragment thereof administered to the subject in each administration is 240 mg.
[E29] The method of any one of E5-E28, wherein the anti-IL-6 receptor antibody or antigen binding fragment thereof is administered to the subject subcutaneously.
[E30] The method of any one of E5-E29, wherein the anti-IL-6 receptor antibody or antigen binding fragment thereof is administered to the subject every two weeks (Q2W) for three times, and thereafter every four weeks (Q4W).
[E31] The method of any one of E1-E30, wherein an immunosuppressive therapy (IST) is administered to the subject concomitantly with the IL-6 inhibitor.
[E32] The method of E31, wherein the IST comprises (i) an immunosuppressive agent selected from the group consisting of azathioprine (AZA), mycophenolate mofetil (MMF), and intravenous (IV) cyclophosphamide; (ii) oral corticosteroid (OCS); or (iii) a combination of (i) and (ii).
[E33] The method of E32, wherein the immunosuppressive agent comprises prednisone, prednisolone or equivalent thereof.
[E34] The method of any one of E1-E33, wherein administering the IL-6 inhibitor to the subject improves the modified Rankin Scale (mRS) score improvement.
[E35] The method of E34, wherein administering the IL-6 inhibitor to the subject
   reduces the time to the modified Rankin Scale (mRS) score improvement without use of rescue therapy
   delays the time to rescue therapy
   delays the time to seizure freedom or cessation of status epilepticus without use of rescue therapy
   improves the Clinical Assessment Scale of Encephalitis (CASE) score
   improves the Montreal Cognitive Assessment (MOCA) total score
   improves the Rey Auditory Verbal Learning Test (RAVLT) score for the subject with an anti-LGI1 antibody and/or
   improves the mRS score for the subject with an anti-NMDAR antibody.
[E36] The method of any one of E1-E35, wherein administering the IL-6 inhibitor to the subject improves the subject's scores of one or more of the followings: modified Rankin Scale (mRS), Clinical Assessment Scale of Encephalitis (CASE), Montreal Cognitive Assessment (MOCA), Rey Auditory Verbal Learning Test (RAVLT), or Columbia-Suicide Severity Rating Scale (C-SSRS).
[E37] The method of any one of E1-E35, wherein administering the IL-6 inhibitor to the subject reduces the subject's modified Rankin Scale (mRS) score, or Clinical Assessment Scale of Encephalitis (CASE) score; or increases the subject's Montreal Cognitive Assessment (MOCA) total score, or Rey Auditory Verbal Learning Test (RAVLT) score.
[E38] The method of any one of E1-E37, wherein the IL-6 inhibitor is administered by a subcutaneous administration device.
[E39] The method of E38, wherein the the subcutaneous administration device is a pre-filled syringe (PFS) optionally comprising a needle safety device (PFS-NSD) or autoinjector (AI).
[F1] A method of reducing risk of relapse of an autoimmune encephalitis (AIE) in a subject, the method comprising administering to the subject an effective amount of an IL-6 inhibitor.
[F2] The method of F1, wherein the IL-6 inhibitor is an anti-IL-6 antibody or antigen-binding fragment thereof, or an anti-IL-6 receptor antibody or antigen binding fragment thereof.
[F3] The method of F1 or F2, wherein the IL-6 inhibitor is an anti-IL-6 receptor antibody or antigen binding fragment thereof.
[F4] The method of any one of F1-F3, wherein the IL-6 inhibitor is a humanized antibody.
[F5] The method of any one of F1-F4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.
[F6] The method of F5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.
[F7] The method of F5 or F6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.
[F8] The method of any one of F5-F7, wherein the IL-6 inhibitor is satralizumab.
[F9] The method of any one of F1-F8, wherein reducing risk of relapse comprises delaying relapse of, reducing frequency of relapse of, reducing severity of relapse of, or reducing the need for rescue therapy for relapse of the autoimmune encephalitis in the subject.
[F10] The method of any one of F1-F9, wherein the subject is positive for anti-N methyl-D aspartic acid receptor (NMDAR) antibody or anti-leucine rich glioma inactivated 1 (LGI1) antibody.
[F11] The method of any one of F1-F10, wherein the autoimmune encephalitis is a disease other than Bickerstaff's brainstem encephalitis, acute disseminated encephalomyelitis, Hashimoto's encephalopathy, primary angiitis of the central nervous system (CNS), and Rasmussen's encephalitis.
[F12] The method of any one of F1-F11, wherein an anti-NMDAR antibody is detected in the cerebral spinal fluid (CSF) from the subject using a cell-based assay.
[F13] The method of any one of F1-F12, wherein the autoimmune encephalitis is anti-NMDAR encephalitis.
[F14] The method of any one of F1-F11, wherein an anti-LGI1 antibody is detected in the serum or cerebral spinal fluid (CSF) from the subject using a cell-based assay.
[F15] The method of F14, wherein the subject has experienced working memory deficits, seizures, or psychiatric symptoms suggesting involvement of the limbic system.
[F16] The method of any one of F1-F11 and F14-F15, the autoimmune encephalitis is anti-LGI1 encephalitis.
[F17] The method of any one of F1-F16, wherein the autoimmune encephalitis is an anti-NMDAR or anti-LGI1 encephalitis.
[F18] The method of any one of F1-F17, wherein the subject is negative for anti-caspr2, anti-IgLON5, anti-DPPX, anti-GABA^{A}, anti-neurexin-3 alpha, and anti-myelin oligodendrocyte glycoprotein (MOG) antibodies.
[F19] The method of any one of F1-F18, wherein the subject is negative for cell surface neuronal antibodies or glial antibodies other than anti-NMDAR and anti-LGI1 antibodies.
[F20] The method of any one of F1-F19, wherein the subject is (i) anti-NMDAR antibody-positive and aged 12 years or older; or (ii) anti-LGI1 antibody-positive and aged 18 years or older.
[F21] The method of any one of F1-F20, wherein the subject is receiving no ongoing chronic immunosuppressive therapy.
[F22] The method of any one of F1-F21, wherein the subject is receiving ongoing treatment with a stable dose of azathioprine (AZA), mycophenolate mofetil (MMF), intravenous (IV) cyclophosphamide, oral corticosteroid (OCS), or a combination of AZA or MMF or IV cyclophosphamide and OCS.
[F23] The method of any one of F5-F22, wherein the subject is determined to have a body weight of less than 40 kg, and the amount of the anti-IL-6 receptor antibody or antigen binding fragment thereof administered to the subject in each administration is 60 mg or 120 mg; or the subject is determined to have a body weight of between 40 and 100 kg, and the amount of the anti-IL-6 receptor antibody or antigen binding fragment thereof administered to the subject in each administration is 120 mg or 180 mg; or the subject is determined to have a body weight of over 100 kg, and the amount of the anti-IL-6 receptor antibody or antigen binding fragment thereof administered to the subject in each administration is 180 mg or 240 mg.
[F24] The method of any one of F5-F22, wherein the subject is determined to have a body weight of less than 40 kg, and the amount of the anti-IL-6 receptor antibody or antigen binding fragment thereof administered to the subject in each administration is 60 mg.
[F25] The method of any one of F5-F22, wherein the subject is determined to have a body weight of less than 40 kg, and the amount of the anti-IL-6 receptor antibody or antigen binding fragment thereof administered to the subject in each administration is 120 mg.
[F26] The method of any one of F5-F22, wherein the subject is determined to have a body weight of between 40 and 100 kg, and the amount of the anti-IL-6 receptor antibody or antigen binding fragment thereof administered to the subject in each administration is 120 mg.
[F27] The method of any one of F5-F22, wherein the subject is determined to have a body weight of between 40 and 100 kg, and the amount of the anti-IL-6 receptor antibody or antigen binding fragment thereof administered to the subject in each administration is 180 mg.
[F28] The method of any one of F5-F22, wherein the subject is determined to have a body weight of over 100 kg, and the amount of the anti-IL-6 receptor antibody or antigen binding fragment thereof administered to the subject in each administration is 180 mg.
[F29] The method of any one of F5-F22, wherein the subject is determined to have a body weight of over 100 kg, and the amount of the anti-IL-6 receptor antibody or antigen binding fragment thereof administered to the subject in each administration is 240 mg.
[F30] The method of any one of F5-F29, wherein the anti-IL-6 receptor antibody or antigen binding fragment thereof is administered to the subject subcutaneously.
[F31] The method of any one of F5-F30, wherein the anti-IL-6 receptor antibody or antigen binding fragment thereof is administered to the subject every two weeks (Q2W) for three times, and thereafter every four weeks (Q4W).
[F32] The method of any one of F1-F31, wherein an immunosuppressive therapy (IST) is administered to the subject concomitantly with the IL-6 inhibitor.
[F33] The method of F32, wherein the IST comprises (i) an immunosuppressive agent selected from the group consisting of azathioprine (AZA), mycophenolate mofetil (MMF), and intravenous (IV) cyclophosphamide; (ii) oral corticosteroid (OCS); or (iii) a combination of (i) and (ii).
[F34] The method of F33, wherein the immunosuppressive agent comprises prednisone, prednisolone or equivalent thereof.
[F35] The method of any one of F1-F34, wherein administering the IL-6 inhibitor to the subject improves the modified Rankin Scale (mRS) score improvement.
[F36] The method of F35, wherein administering the IL-6 inhibitor to the subject
   reduces the time to the modified Rankin Scale (mRS) score improvement without use of rescue therapy
   delays the time to rescue therapy
   delays the time to seizure freedom or cessation of status epilepticus without use of rescue therapy
   improves the Clinical Assessment Scale of Encephalitis (CASE) score
   improves the Montreal Cognitive Assessment (MOCA) total score
   improves the Rey Auditory Verbal Learning Test (RAVLT) score for the subject with an anti-LGI1 antibody and/or
   improves the mRS score for the subject with an anti-NMDAR antibody.
[F37] The method of any one of F1-F36, wherein administering the IL-6 inhibitor to the subject improves the subject's scores of one or more of the followings: modified Rankin Scale (mRS), Clinical Assessment Scale of Encephalitis (CASE), Montreal Cognitive Assessment (MOCA), Rey Auditory Verbal Learning Test (RAVLT), or Columbia-Suicide Severity Rating Scale (C-SSRS).
[F38] The method of any one of F1-F36, wherein administering the IL-6 inhibitor to the subject reduces the subject's modified Rankin Scale (mRS) score, or Clinical Assessment Scale of Encephalitis (CASE) score; or increases the subject's Montreal Cognitive Assessment (MOCA) total score, or Rey Auditory Verbal Learning Test (RAVLT) score.
[F39] The method of any one of E1-E38, wherein the IL-6 inhibitor is administered by a subcutaneous administration device.
[F40] The method of E39, wherein the the subcutaneous administration device is a pre-filled syringe (PFS) optionally comprising a needle safety device (PFS-NSD) or autoinjector (AI).

### Advantageous Effects of Invention

The present invention can provide a medicament (a pharmaceutical composition) comprising satralizumab for treating, preventing, preventing relapse of, or reducing risk of relapse of an autoimmune encephalitis, such as anti-NMDAR encephalitis and anti-LGI1 encephalitis.

### BRIEF DESCRIPTION OF DRAWINGS

FIG. 1 shows the primary treatment period of the study schema of Phase III, randomized, double-blind, placebo-controlled, multicenter basket study to evaluate the efficacy, safety, pharmacokinetics, and pharmacodynamics of satralizumab in patients with anti-N methyl-D aspartic acid receptor (NMDAR) and anti-leucine rich glioma inactivated 1 (LGI1) encephalitis. AIE=autoimmune encephalitis; LGI1=leucine-rich glioma-inactivated 1; NMDAR=N-methyl-D-aspartic acid receptor; PK=pharmacokinetic; Q4W=every 4 weeks; R=randomization. Notes: Week 0 baseline assessments will be performed predose. The specific dose of satralizumab (or matching placebo) is based on individual participant's body weight, as follows: less than 40 kg: 60 mg or 120 mg Q4W; between 40 and 100 kg: 120 mg or 180 mg Q4W; more than 100 kg: 180 mg or 240 mg Q4W.

FIG. 2 shows the dependencies of steady-state exposure parameters and receptor occupancy on body weight following 60 mg (less than 40 kg), 120 mg (between 40 and 100 kg), or 180 mg (more than 100 kg) Q4W dosing. Note: Predictions for Cₘₐₓ, C_{trough}, and RO are shown from left to right, respectively. The simulations are based on 2000 individuals. Points are simulated data assuming ADA positivity in a similar proportion of participants as was observed in NMOSD studies. For body weight <40 kg, no ADA is assumed. Dashed horizontal lines have been added for reference. ADA=anti-drug antibody; Cmax=maximum concentration observed; Ctr=steady-state concentration at the end of a dosing interval; NMOSD=neuromyelitis optica spectrum disorder; RO=receptor occupancy; Q4W=every 4 weeks.

### DESCRIPTION OF EMBODIMENTS

The present invention relates to a medicament (a pharmaceutical composition) for treating an autoimmune encephalitis (AIE), or for reducing risk of relapse in an autoimmune encephalitis (AIE), including but not limited to, anti-N-methyl-D-aspartic acid receptor (NMDAR) encephalitis and anti-leucine-rich glioma-inactivated 1 (LGI1) encephalitis, in a subject, comprising an IL-6 inhibitor as an active ingredient. A relapse is defined as a new clinical episode (new or worsening, acute symptoms and clinical signs of AIE appearing, e.g., a month later after the last attack).

In another aspect, the present invention also relates to use of an IL-6 inhibitor in the preparation of a medicament for treating an autoimmune encephalitis (AIE), including but not limited to, anti-N-methyl-D-aspartic acid receptor (NMDAR) encephalitis and anti-leucine-rich glioma-inactivated 1 (LGI1) encephalitis, or reducing risk of relapse in an autoimmune encephalitis (ATE) in a subject.

In yet another aspect, the present invention relates to an IL-6 inhibitor for use in treating an autoimmune encephalitis (AIE), including but not limited to, anti-N-methyl-D-aspartic acid receptor (NMDAR) encephalitis and anti-leucine-rich glioma-inactivated 1 (LGI1) encephalitis, or reducing risk of relapse in an autoimmune encephalitis (AIE) in a subject.

Furthermore, the present invention also relates to a kit for treating an autoimmune encephalitis (AIE), including but not limited to, anti-N-methyl-D-aspartic acid receptor (NMDAR) encephalitis and anti-leucine-rich glioma-inactivated 1 (LGI1) encephalitis, or reducing risk of relapse in an autoimmune encephalitis (AIE), in a subject, which comprises a pharmaceutical composition comprising an IL-6 inhibitor, and a package insert or label instructing administration of the pharmaceutical composition to a subject.

Moreover, the present invention also relates to a method of treating a subject having an autoimmune encephalitis (AIE), including but not limited to, anti-N-methyl-D-aspartic acid receptor (NMDAR) encephalitis and anti-leucine-rich glioma-inactivated 1 (LGI1) encephalitis, or reducing risk of relapse in an autoimmune encephalitis (AIE), in a subject, the method comprising administering to the subject an effective amount of an IL-6 inhibitor.

An “IL-6 inhibitor” of the present disclosure is a substance that blocks signal transduction by IL-6 and inhibits the biological activities of IL-6. The IL-6 inhibitor is preferably a substance that inhibits binding between IL-6 and IL-6 receptor and/or between the IL-6/IL-6 receptor complex and gp130. Examples of an IL-6 inhibitor of the present disclosure include, but are not particularly limited to, an anti-IL-6 antibody or antigen binding fragment thereof, an anti-IL-6 receptor antibody or antigen binding fragment thereof, an anti-gp130 antibody or antigen binding fragment thereof, an IL-6 variant, a soluble IL-6 receptor variant, or a partial peptide of IL-6 or IL-6 receptor, and a low-molecular-weight substance showing a similar activity. Examples of an IL-6 inhibitor of the present disclosure may be preferably an anti-IL-6 antibody or antigen-binding fragment thereof, or an anti-IL-6 receptor antibody or antigen binding fragment thereof, more preferably an anti-IL-6 receptor antibody or antigen binding fragment thereof, optionally a humanized antibody.

In some embodiments of the present disclosure, the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10. In certain embodiments in the present disclosure, the anti-IL-6 receptor antibody or antigen binding fragment thereof comprises a VH comprising the amino acid sequence of SEQ ID NO: 1 and a VL comprising the amino acid sequence of SEQ ID NO: 2. In certain embodiments in the present disclosure, the IL-6 inhibitor is an anti-IL-6 receptor antibody comprising a heavy chain comprising the amino acid sequence of SEQ ID NO: 3 and a light chain comprising the amino acid sequence of SEQ ID NO: 4. In certain embodiments in the present disclosure, the IL-6 inhibitor is satralizumab. In some embodiments of the present disclosure, the term “satralizumab” is an anti-IL-6 receptor antibody that comprises a heavy-chain CDR1 comprising the amino acid sequence of SEQ ID NO: 5, a heavy-chain CDR2 comprising the amino acid sequence of SEQ ID NO: 6, a heavy-chain CDR3 comprising the amino acid sequence of SEQ ID NO: 7, a light-chain CDR1 comprising the amino acid sequence of SEQ ID NO: 8, a light-chain CDR2 comprising the amino acid sequence of SEQ ID NO: 9, and a light-chain CDR3 comprising the amino acid sequence of SEQ ID NO: 10; preferably comprises a heavy-chain variable region comprising the amino acid sequence of SEQ ID NO: 1 and a light-chain variable region comprising the amino acid sequence of SEQ ID NO: 2; and most preferably comprises a heavy chain comprising the amino acid sequence of SEQ ID NO: 3 and a light chain comprising the amino acid sequence of SEQ ID NO: 4.

In certain embodiments in the present disclosure, the autoimmune encephalitis (AIE) includes disorders that are associated with an identifiable etiological driver, usually a tumor, and disorders that are regarded as idiopathic. In the paraneoplastic immune-mediated encephalitis syndromes, those may be associated with antibodies against intracellular neuronal proteins (onconeuronal proteins such as anti-Hu). In one aspect, the AIE is delineated by the identification of antibodies against neuronal cell surface and synaptic proteins such as Hu (ANNA1), Ma2, GAD, N-terminal enolase (NAE), NMDA receptor, AMPA receptor, GABA^{A} receptor, GABA^{B} receptor, mGluR5, dopamine D2 receptor, anti-leucine-rich glioma-inactivated 1 (LGI1), CASPR2, and DPPX (Graus et al., Lancet Neurol., 15, 391-404, 2016; Lancaster et al., Neurology, 77, 179-189, 2011), which are thought to be directly pathogenic for the AIE. The identification of those autoantibodies may help to delineate a population with better response to the present invention. In the present disclosure, the AIE includes any AIE subtypes which one or more of the autoantibodies are thought to be one of the pathogenic cause, and anti-N-methyl-D-aspartic acid receptor (NMDAR) encephalitis and anti-leucine-rich glioma-inactivated 1 (LGI1) encephalitis represent two of the most common and best characterized AIE (Leypoldt F, Wandinger K-P, Bien C, et al., Eur Neurol Rev 2013; 8:31-7). In one aspect, the AIE in the present disclosure also includes Bickerstaff's brainstem encephalitis, acute disseminated encephalomyelitis, Hashimoto's encephalopathy, primary angiitis of the central nervous system (CNS), and Rasmussen's encephalitis. In the present disclosure, the anti-NMDAR encephalitis and anti-LGI1 encephalitis are described as more specific embodiments.

In the present disclosure, “a subject”, which may be referred as “a patient”, has the above-described autoimmune encephalitis (AIE) such as anti-N-methyl-D-aspartic acid receptor (NMDAR) encephalitis and anti-leucine-rich glioma-inactivated 1 (LGI1) encephalitis. In one aspect, the subject is positive for anti-N methyl-D aspartic acid receptor (NMDAR) antibody or anti-leucine rich glioma inactivated 1 (LGI1) antibody. In one aspect, whether or not, the subject is positive for anti-NMDAR or anti-LGI1 is detected in the cerebral spinal fluid (CSF) from the subject using a cell-based assay. In one aspect, the subject having anti-NMDAR or LGI1 encephalitis is negative for anti-caspr2, anti-IgLON5, anti-DPPX, anti-GABA^{A}, anti-neurexin-3 alpha, and anti-myelin oligodendrocyte glycoprotein (MOG) antibodies. In one aspect, the subject having anti-NMDAR or LGI1 encephalitis is negative for cell surface neuronal antibodies or glial antibodies other than anti-NMDAR and anti-LGI1 antibodies. In one aspect, the subject having anti-NMDAR or LGI1 encephalitis has experienced working memory deficits, seizures, or psychiatric symptoms suggesting involvement of the limbic system. In another aspect, the subject having anti-NMDAR or LGI1 encephalitis has received, has not received, is receiving, or is not receiving chronic immunosuppressive therapy (IST). In one aspect, the subject having anti-NMDAR or LGI1 encephalitis has, has received, has not received, is receiving, or is not receiving treatment with a stable dose of azathioprine (AZA), mycophenolate mofetil (MMF), intravenous (IV) cyclophosphamide, oral corticosteroid (OCS), or a combination of AZA or MMF or IV cyclophosphamide and OCS. In one aspect, the subject having anti-NMDAR or LGI1 encephalitis (i) anti-NMDAR antibody-positive and aged 12 years or older; or (ii) anti-LGI1 antibody-positive and aged 18 years or older.

In certain embodiments, the medicament or the pharmaceutical composition of the present invention is used in combination with an immunosuppressive therapy (IST). In certain embodiments, the IST is one or more immunosuppressive agents selected from the group consisting of azathioprine (AZA), mycophenolate mofetil (MMF), and intravenous (IV) cyclophosphamide; (ii) oral corticosteroid (OCS); or (iii) a combination of (i) and (ii).

In certain embodiments, the medicament or the pharmaceutical composition of the present invention can delay the time from an administration of the IL-6 inhibitor to the first occurrence of a relapse of an autoimmune encephalitis (AIE), including but not limited to, anti-N-methyl-D-aspartic acid receptor (NMDAR) encephalitis and anti-leucine-rich glioma-inactivated 1 (LGI1) encephalitis. In certain embodiments, the medicament or the pharmaceutical composition of the present invention can further reduce one or more of the followings:
(a) the rate of relapses of the autoimmune encephalitis (AIE);
(b) the rate of active lesions on MRI of the neuroaxis;
(c) the proportion of subjects receiving rescue therapy; or
(d) the rate of inpatient hospitalizations.

In the present disclosure, the term “treatment” is used with the meaning that even if complete remission of the AIE is not achieved, alleviating or improving symptoms to a level at which minimal manifestations (MM) can be maintained, or maintaining such a state, is also included in the “treatment” of the AIE.

In the present disclosure, efficacy, safety, pharmacokinetics and/or pharmacodynamics of IL-6 inhibitor such as satralizumab for the treatment of AIE may be assessed based on one or more aspects and/or scores as described below.
i. Improving the modified Rankin Scale (mRS) score improvement.
ii. Reducing the time to the modified Rankin Scale (mRS) score improvement without use of rescue therapy.
iii. Delaying the time to rescue therapy.
iv. Delaying the time to seizure freedom or cessation of status epilepticus without use of rescue therapy.
v. Improving the Clinical Assessment Scale of Encephalitis (CASE) score.
vi. Improving the Montreal Cognitive Assessment (MOCA) total score.
vii. Improving the Rey Auditory Verbal Learning Test (RAVLT) score for the subject with an anti-LGI1 antibody.
viii. Improving the mRS score for the subject with an anti-NMDAR antibody.
viii. Improving Modified Fatigue Impact Scale (MFIS).
ix. Improving Montreal Cognitive Assessment (MOCA).
x. Improving Rey Auditory Verbal Learning Test (RAVLT).
xi. Improving EQ-5D-5L.
xii. Improving Columbia-Suicide Severity Rating Scale (C-SSRS).
xiii. Improving Beck Depression Inventory (BDI II).

The given period of applying the present invention (e.g., administering a medicament or a pharmaceutical composition of the present invention) for evaluating efficacy is not particularly limited and includes 1 week, 2 weeks, 4 weeks, 8 weeks, 12 weeks, 24 weeks, 48 weeks, 1 year, 2 years, 3 years, 4 years, and 5 years, and the period may be shorter or longer than the exemplified period.

In the present invention, a subject (e.g., a patient) having the autoimmune encephalitis (AIE) may receive a treatment of the present invention (e.g., a medicament, a pharmaceutical composition, a method, or the like), e.g., every two weeks (Q2W) for three times (i.e., at time zero and again at 2 weeks and 4 weeks), and thereafter every four weeks (Q4W). In some embodiments, the subject (e.g., patient) can receive the anti-IL-6 receptor antibody (e.g., satralizumab) or antigen binding fragment thereof contained in a medicament or a composition of the present invention via subcutaneous administration route.

In addition to a treatment of the AIE in a subject of the present invention, the present invention is also used for reducing risk of relapse in a relapsing the AIE in the subject. In the present invention, reduction of the risk of relapse includes but is not limited to delaying relapse of, reducing frequency of relapse of, or reducing severity of relapse of, or reducing the need for rescue therapy for relapse of the AIE.

An anti-IL-6 receptor antibody or antigen binding fragment thereof used in the present invention binds to an IL-6 receptor, inhibits the binding of IL-6 to an IL-6 receptor, blocks signal transduction by IL-6, and inhibits the biological activities of IL-6.

An anti-IL-6 receptor antibody used in the present invention can be obtained using known methods. In particular, an anti-IL-6 receptor antibody used in the present invention is preferably a monoclonal antibody derived from a mammal. Monoclonal antibodies derived from a mammal include those produced by a hybridoma and those produced by a host that has been transformed with an expression vector containing an antibody gene using genetic engineering methods.

Preferred examples of an “IL-6 receptor antibody” in the present invention include humanized anti-IL-6 receptor antibodies produced by modifying the variable and constant regions of tocilizumab, specifically, antibodies that comprise a heavy-chain CDR1 comprising the amino acid sequence of SEQ ID NO: 5, a heavy-chain CDR2 comprising the amino acid sequence of SEQ ID NO: 6, a heavy-chain CDR3 comprising the amino acid sequence of SEQ ID NO: 7, a light-chain CDR1 comprising the amino acid sequence of SEQ ID NO: 8, a light-chain CDR2 comprising the amino acid sequence of SEQ ID NO: 9, and a light-chain CDR3 comprising the amino acid sequence of SEQ ID NO: 10.

More preferred antibodies in the present invention include antibodies that comprise a heavy-chain variable region comprising the amino acid sequence of SEQ ID NO: 1 and a light-chain variable region comprising the amino acid sequence of SEQ ID NO: 2. Still more preferred are antibodies that comprise a heavy chain comprising the amino acid sequence of SEQ ID NO: 3 (heavy chain of satralizumab (generic name); SA237(private name)) and a light chain comprising the amino acid sequence of SEQ ID NO: 4 (light chain of satralizumab). Satralizumab (private name: SA237) is particularly preferred.

Governmental marketing approval of satralizumab has been obtained in many countries including Japan, United States, and Europe based on the indication “prevention of relapses of neuromyelitis optica spectrum disorder (including neuromyelitis optica)”. The safety profiles identified during the international joint phase III clinical trials (SA-307JG/BN40898 study and SA-309JG/BN40900 study) targeting a population of patients with neuromyelitis optica spectrum disorder (NMOSD) and/or neuromyelitis optica (NMO) were mostly favorable. No death case was reported. The percentage of patients who experienced severe adverse events in the satralizumab group was about the same as that in the placebo group. There was no big difference between the two groups on the frequency of adverse events that led to discontinuation of administration of the test drug, or on the frequency of adverse events that led to drug withdrawal. Safety profiles were similar between the SA-309JG study, which was a single-agent test, and the SA-307JG study, which was a combined test with preexisting therapy (oral steroids and/or immunosuppressive agents).

Such antibodies can be obtained according to the methods described in WO2010/035769, WO2010/107108, WO2010/106812, and such. Specifically, antibodies can be produced using genetic recombination techniques known to those skilled in the art, based on the sequence of the above-mentioned IL-6 receptor antibody (see, for example, Borrebaeck C A K and Larrick J W, THERAPEUTIC MONOCLONAL ANTIBODIES, Published in the United Kingdom by MACMILLAN PUBLISHERS LTD, 1990). A recombinant antibody can be obtained by cloning a DNA encoding the antibody from a hybridoma or an antibody-producing cell such as an antibody-producing sensitized lymphocyte, inserting the DNA into an appropriate vector, and introducing the vector into a host (host cell) to produce the antibody.

Such antibodies can be isolated and purified using isolation and purification methods conventionally used for antibody purification, without limitation. For example, the antibodies can be isolated and purified by appropriately selecting and combining column chromatography, filtration, ultrafiltration, salting-out, solvent precipitation, solvent extraction, distillation, immunoprecipitation, SDS-polyacrylamide gel electrophoresis, isoelectric focusing, dialysis, recrystallization, and such.

The antibodies used in the present invention may be conjugate antibodies that are bound to various molecules such as polyethylene glycol (PEG), radioactive substances, and toxins. Such conjugate antibodies can be obtained by chemically modifying the obtained antibodies. Methods for antibody modification have been already established in this field. Accordingly, the term “antibody” in the present invention encompasses such conjugate antibodies.

The antibodies used in the present invention may be antibody fragments (also referred to as an antigen binding fragment of the antibody) or modified products thereof, as long as they can be suitably used in the present invention. For example, antibody fragments include Fab, F(ab′)2, Fv, and single chain Fv (scFv) in which the Fvs of the H and L chains are linked via an appropriate linker.

Specifically, the antibody fragments are produced by treating antibodies with enzymes such as papain or pepsin, or alternatively, by constructing genes encoding these antibody fragments and introducing them into expression vectors, and then expressing the vectors in appropriate host cells (see, for example, Co, M. S. et al., J. Immunol. (1994) 152, 2968-2976; Better, M. & Horwitz, A. H., Methods in Enzymology (1989) 178, 476-496; Plueckthun, A. & Skerra, A., Methods in Enzymology (1989) 178, 497-515; Lamoyi, E., Methods in Enzymology (1989) 121, 652-663; Rousseaux, J. et al., Methods in Enzymology (1989) 121, 663-666; and Bird, R. E. et al., TIBTECH (1991) 9, 132-137).

An scFv can be obtained by linking the H-chain V region and the L-chain V region of an antibody. In this scFv, the H-chain V region and the L-chain V region are linked via a linker, preferably via a peptide linker (Huston, J. S. et al., Proc. Natl. Acad. Sci. USA (1988) 85, 5879-5883). The V regions of the H and L chains in an scFv may be derived from any of the antibodies described above. Peptide linkers for linking the V regions include, for example, an arbitrary single chain peptide consisting of 12 to 19 amino acid residues.

A DNA encoding an scFv can be obtained by amplifying a DNA portion that encodes the desired amino acid sequence in template sequences with PCR using a primer pair which defines the termini of the portion, wherein a DNA encoding an H chain or an H-chain V region and a DNA encoding an L chain or an L-chain V region of the afore-mentioned antibodies are used as the templates, and then further amplifying the amplified DNA portion with a DNA that encodes a peptide linker portion and a primer pair that defines both ends of the linker so that it may be linked to each of the H and L chains.

Once an scFv-encoding DNA has been prepared, an expression vector comprising the DNA and a host transformed with the expression vector can be obtained according to conventional methods. In addition, an scFv can be obtained according to conventional methods by using the host.

Similar to the above, the antibody fragments can be produced by obtaining their genes, expressing them, and then using a host.

In the present invention, “as an active ingredient” means that the ingredient is contained in the pharmaceutical composition as a primal active ingredient, and the content thereof is not limited unless specifically indicated, as long as the antibodies or antigen binding fragments thereof used for the present invention are included as medicinal ingredients.

The dose of an anti-TL-6 receptor antibody or antigen binding fragment thereof contained in a medicament or a composition of the present invention is not particularly limited, and examples include 50 to 800 mg of antibody per administration, preferably 60 to 240 mg of antibody, and more preferably 60 mg, 120 mg, 180 mg, or 240 mg of antibody per administration. The dose of an anti-IL-6 receptor antibody or antigen binding fragment thereof contained in a medicament or a composition of the present invention may vary depending on the patient's body weight. In certain embodiments of the present invention, a suitable dose of the anti-IL-6 receptor antibody or antigen binding fragment for a subject with a body weight of less than 40 kg is 60 mg or 120 mg; a suitable dose for a subject with a body weight between 40 kg and 100 kg is 120 mg or 180 mg; and a suitable dose for a subject with a body weight of over 100 kg is 180 mg or 240 mg. A medicament or a composition comprising an anti-IL-6 receptor antibody or antigen binding fragment thereof of the present invention is administered to a subject via any route, including but not limited to subcutaneously, intravenously, intramuscularly, and by infusion. A preferred embodiment is subcutaneous administration.

In certain embodiments of the present invention, two or more sequential doses of an anti-IL-6 receptor antibody or antigen binding fragment thereof contained in a medicament or a composition of the present invention is administered to a subject during an initial period, wherein the doses administered during the initial period are spaced by a first dosing interval (also referred to the dosing interval that is shorter than the routine dosing interval), for example 20 weeks, 8 weeks, 4 weeks, or two weeks; and after the final dose administration of the initial period, waiting a second dosing interval that is longer than the first dosing interval and then administering a dose of an anti-IL-6 receptor antibody or antigen binding fragment thereof contained in a medicament or a composition of the present invention to a human patient, wherein optionally multiple consecutive doses are administered after the final dose administration of the initial period, and are spaced by the second dosing interval (also referred to as a “routine dosing interval”) that is not particularly limited except that it is longer than the first dosing interval. Examples of the second dosing interval include 1 day to 24 weeks, preferably 2 weeks to 8 weeks, more preferably 3 to 5 weeks, and even more preferably 4 weeks).

In certain embodiments of the present invention, an anti-IL-6 receptor antibody or antigen binding fragment thereof contained in a medicament or a composition of the present invention is administered to a subject every two weeks (Q2W) for three times, and thereafter every four weeks (Q4W).

The preferred administration schedule for an anti-IL-6 receptor antibody or antigen binding fragment thereof contained in a medicament or a composition of the present invention can be adjusted, for example, by appropriately extending the administration interval by monitoring the conditions of the disease and changes in the blood test values.

The present invention also provides an article of manufacture such as a kit, a device, and the like for use in a method of the present invention, which contains a pharmaceutical composition or a medicament of the present invention. The pharmaceutical composition or a medicament of the present invention comprises an IL-6 inhibitor as described herein. The article of manufacture may be packaged with an additional pharmaceutically acceptable carrier or medium, or instruction manual describing how to use the kits, etc.

In one embodiment, the article of manufacture comprises a container and a label on or a package insert associated with the container. Suitable containers include, for example, bottles, vials, syringes (including a prefilled syringe and an autoinjector), IV solution bags, etc. The containers may be formed from a variety of materials such as glass or plastic. In one embodiment, the container holds a composition which is by itself or combined with another composition effective for treating, preventing and/or diagnosing the condition and may have a sterile access port (for example the container may be a syringe, autoinjector, an intravenous solution bag or a vial having a stopper pierceable by a hypodermic injection needle). At least one active ingredient in the composition is an IL-6 inhibitor, preferably an anti-IL-6 receptor antibody, and more preferably satralizumab as described in the present disclosure.

In one embodiment, a device as the article of manufacture of the present invention as described above may be a prefilled syringe (PFS) optionally comprising a needle safety device (PFS-NSD) for injection via any administration route such as intravenously, subcutaneously, or the like, which comprises a fixed dose of an IL-6 inhibitor, preferably an anti-IL-6 receptor antibody, and more preferably satralizumab as described in the present disclosure in a pharmaceutically acceptable excipient. In another embodiment, the device may be an autoinjector (AI) for subcutaneous administration which comprises a fixed dose of an IL-6 inhibitor, preferably an anti-IL-6 receptor antibody, and more preferably satralizumab as described in the present disclosure in a pharmaceutically acceptable excipient. In certain embodiments, the device is a subcutaneous administration device, such as a prefilled syringe (PFS) and autoinjector (AI), which may comprise 60 mg, 120 mg, 180 mg, or 240 mg of satralizumab. In one embodiment, the subcutaneous administration device is a prefilled syringe comprising a needle safety device (PFS-NSD) which comprises 60 mg (for example 60 mg/mL) of satralizumab for delivering 60 mg or 180 mg doses to a subject. In another embodiment, the subcutaneous administration device is a prefilled syringe comprising a needle safety device (PFS-NSD) which comprises 120 mg (for example 60 mg/0.5 mL) of satralizumab for delivering 120 mg, 180 mg, or 240 doses to the subject. In other embodiments, the subcutaneous administration device is an autoinjector (AI) which comprises 120 mg, 180 mg, or 240 mg of satralizumab.

In the present invention, the label or package insert indicates that the pharmaceutical composition or medicament is used for treating the condition of choice. Moreover, the article of manufacture may comprise (a) a first container with a composition contained therein, wherein the composition comprises an IL-6 inhibitor, preferably an anti-IL-6 receptor antibody, and more preferably satralizumab as described above; and (b) a second container with a composition contained therein, wherein the composition comprises a further therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Package Insert

The term “package insert” is used to refer to instructions customarily included in commercial packages of therapeutic products, that contain information about the indications, usage, dosage, administration, combination therapy, contraindications and/or warnings concerning the use of such therapeutic products.

A pharmaceutical composition or a medicament of the present invention can be formulated to produce freeze-dried formulations or solution formulations by mixing, if necessary, with suitable pharmaceutically acceptable carriers, vehicles, and such. The suitable pharmaceutically acceptable carriers and vehicles include, for example, sterilized water, physiological saline, stabilizers, excipients, antioxidants (such as ascorbic acid), buffers (such as phosphate, citrate, histidine, and other organic acids), antiseptics, surfactants (such as PEG and Tween), chelating agents (such as EDTA), and binders. Other low-molecular-weight polypeptides, proteins such as serum albumin, gelatin, and immunoglobulins, amino acids such as glycine, glutamine, asparagine, glutamic acid, aspartic acid, methionine, arginine, and lysine, sugars and carbohydrates such as polysaccharides and monosaccharides, and sugar alcohols such as mannitol and sorbitol may also be contained in the formulation. When preparing an aqueous solution for injection, physiological saline and isotonic solutions comprising glucose and other adjuvants such as D-sorbitol, D-mannose, D-mannitol, and sodium chloride may be used; and appropriate solubilizers such as alcohol (for example, ethanol), polyalcohols (such as propylene glycol and PEG), and nonionic surfactants (such as polysorbate 80, polysorbate 20, poloxamer 188, and HCO-50) may be used in combination. By mixing hyaluronidase into the formulation, a larger fluid volume can be administered subcutaneously (Expert Opin. Drug Deliv. 2007 July; 4(4): 427-40). Furthermore, syringes may be prefilled with the pharmaceutical composition of the present invention. Solution formulations can be prepared according to the method described in WO2011/090088.

If necessary, a pharmaceutical composition or a medicament of the present invention may be encapsulated in microcapsules (e.g., those made of hydroxymethylcellulose, gelatin, and poly(methylmethacrylate)), or incorporated into colloidal drug delivery systems (e.g., liposomes, albumin microspheres, microemulsions, nanoparticles, and nanocapsules) (see, for example, “Remington's Pharmaceutical Science 16th edition”, Oslo Ed. (1980)). Methods for preparing the pharmaceutical agents as controlled-release pharmaceutical agents are also known, and such methods may be applied to the pharmaceutical compositions of the present invention (Langer et al., J. Biomed. Mater. Res. 15: 267-277 (1981); Langer, Chemtech. 12: 98-105 (1982); U.S. Pat. No. 3,773,919; European Patent Application Publication No. EP 58,481; Sidman et al., Biopolymers 22: 547-556 (1983); and EP 133,988).

An anti-IL-6 receptor antibody or antigen binding fragment thereof contained in a medicament or a composition of the present invention can be administered to a patient via any appropriate route. For example, it can be administered to a patient intravenously by bolus injection or by continuous infusion, intramuscularly, intraperitoneally, intracerebrospinally, transdermally, subcutaneously, intraarticularly, sublingually, intrasynovially, orally, by inhalation, locally, or externally, for a certain period of time. Intravenous administration or subcutaneous administration is preferred. In certain embodiments of the present invention, an anti-IL-6 receptor antibody or antigen binding fragment thereof contained in a medicament or a composition of the present invention is administered to a subject subcutaneously.

All prior art references cited herein are incorporated by reference into the present specification.

### EXAMPLES

Herein below, the present invention will be specifically described with reference to the Examples, but it is not to be construed as being limited thereto.

### Example 1: Preparation of Satralizumab (SA237)

An antibody with the generic name satralizumab (and a private name of SA237), which is an IL-6 receptor antibody described in the patent document WO 2010/035769 as comprising a heavy chain having the amino acid sequence of SEQ ID NO: 26 (SEQ ID NO: 3 in the present specification) and a light chain having the amino acid sequence of SEQ ID NO: 29 (SEQ ID NO: 4 in the present specification)), was prepared according to the description of that patent document. The amino acid sequence of the heavy chain variable region is shown in SEQ ID NO: 1, and the amino acid sequence of the light chain variable region is shown in SEQ ID NO: 2. Using the prepared antibody, a subcutaneous administration preparation was prepared by the method described in the patent document WO 2011/090088.

### Example 2: Phase III, Randomized, Double-Blind, Placebo-Controlled, Multicenter Basket Study to Evaluate the Efficacy, Safety, Pharmacokinetics, and Pharmacodynamics of Satralizumab in Patients with Anti-N Methyl-D Aspartic Acid Receptor (NMDAR) or Anti-Leucine Rich Glioma Inactivated 1 (LGI1) Encephalitis

### 1. Introduction

### 1.1 Study Rationale

The purpose of this study is to assess the efficacy, safety, pharmacokinetics, and pharmacodynamics of satralizumab in participants with anti-N-methyl-D-aspartic acid receptor (NMDAR) and anti-leucine-rich glioma-inactivated 1 (LGI1) encephalitis.

Although the NMDAR and LGI1 encephalitides are distinct and diagnostically distinguishable disease subtypes, both share the core clinical features of seizures and cognitive impairments; therefore, it is appropriate to apply the same study endpoints, study duration, and study design to each subtype but to analyze outcomes in each subtype independently. In order to balance these considerations, participants with either of these two encephalitides will be clearly separated into distinct cohorts, each with its own placebo control and analysis.

Current treatment of these disorders exclusively use off-label immune therapies and is based on expert opinion, retrospective case series, and open-label studies. Several unmet needs exist, including the frequent occurrence of long-term cognitive deficits, insufficient seizure control, frequent dependence on high-dose corticosteroids, and faster acting but durable immunotherapy. There is a need for prospectively generated evidence-based treatments to meaningfully lessen the acute and long-term con-sequences of these disorders.

### 1.2 Benefit-Risk Assessment

The purpose of this study is to assess the efficacy and safety of satralizumab, an IL-6R blocker, to address a significant unmet medical need in participants with AIE.

Although satralizumab has not been tested previously in patients with either anti-NMDAR- or anti-LGI1-mediated encephalitis, nonclinical and clinical data suggest that IL-6 plays a key role in the pathophysiology of these diseases. IL-6 is a pro-inflammatory cytokine with pleiotropic functions including induction of the differentiation and proliferation of pro-inflammatory Th17 cells and plasmablasts, and plasma cell maturation. IL-6R blockade has the potential to modulate the immunopathogenic mechanisms of ATE irrespective of autoantibody type. Processes regulated by IL-6R signaling, such as B- and T-cell differentiation, B cell proliferation, and regulation of blood-brain barrier have been suggested to have a role in the pathogenesis of both anti-NMDAR- (Armangue et al. 2018; Martinez-Hernandez et al. 2011; Bien et al. 2012; Leypoldt et al. 2015; Ding et al. 2018) and anti-LGI1-mediated AIE (Helmstaedter et al. 2021).

Nonclinical data generated with IL-6 knockout mice and with IL-6 neutralizing antibodies demonstrate a clear role of IL-6 in the pathogenesis of experimental AIE. In rats administered NMDAR antibodies, cerebral infusion of IL-6 was associated with worsening of NMDAR-mediated excitatory postsynaptic currents and was accompanied by heightened impairment of memory and learning performance (Wang et al. 2019). In addition, an increase in CSF IL-6 levels have been observed in patients with NMDAR encephalitis and in patients with new-onset refractory status epilepticus, a condition commonly associated with AIE (June et al. 2018; Kirmani et al. 2018).

Furthermore, clinical case series suggest a benefit associated with IL-6R antagonism in patients with AIE and associated symptoms. A retrospective study demonstrated significant benefit of anti-IL-6R treatment (tocilizumab) administered to adult patients with varying antibody subtypes (including anti-NMDAR and anti-LGI1) with incomplete response to prior rituximab (Lee et al. 2016a; Lee et. al 2016b). Similar responses were seen in children (Randell et al. 2018). IL-6R antagonism was also associated with significant clinical improvement when administered to new onset NMDAR- and CASPR2-positive AIE patients with recent symptom onset (Lee et. al. 2020; Krogias et al. 2013). Similar beneficial effects were observed in patients with “Probable AIE” (i.e., meeting AIE clinical criteria, but with no clear autoantibody association) (Lee et. al. 2020). Finally, IL-6R inhibition is associated with the cessation of refractory status epilepticus, a life-threatening condition observed in as many as 30% of patients with AIE in acute care (Cadena et al. 2017; June et al. 2018).

Satralizumab has demonstrated efficacy and safety and is indicated for another autoantibody-mediated disease (i.e., NMOSD) as monotherapy or as an add-on to immunosuppressive therapy (IST; i.e., oral corticosteroid [OCS], azathioprine, or mycophenolate mofetil). The double-blind period of the Phase III studies in NMOSD included a total of 178 participants. Of the 178 participants, 104 participants were treated with a satralizumab dose of 120 mg subcutaneously every 4 weeks (Q4W) and 74 participants with placebo. Overall, satralizumab as monotherapy or in combination with IST was well tolerated by participants with NMOSD.

All currently available treatment options for AIE (including high-dose corticosteroids and cyclophosphamide) carry substantial potential safety risks. There is no evidence from randomized controlled trials to support treatment decisions. In addition, not all patients respond to currently used medications, and deficiencies remain, including cognitive deficits, insufficient seizure resolution, dependence on high-dose corticosteroids and a need for durable, faster acting agents. The proposed trial of satralizumab will address these unmet needs and establish the effectiveness of satralizumab in patients with NMDAR or LGI1 encephalitis. Taking into account the potential for efficacy in patients for which no approved drug exists, the safety profile for satralizumab, and the risk-mitigation measures for the study, the benefit-risk ratio is expected to be acceptable for satralizumab in the treatment of both NMDAR and LGI1 encephalitis.

### 2. Objectives and Endpoints

This study will evaluate the efficacy, safety, pharmacokinetics, and pharmacodynamics of satralizumab compared with placebo in each of the following cohorts:
NMDAR AIE cohort: adults and adolescents with definite or probable NMDAR encephalitis
LGI1 AIE cohort: adults with LGI1 encephalitis

For efficacy analyses, each cohort will be treated as a separate population and will have independent Type I error control at a 5% significance level.

Specific objectives and corresponding endpoints for both cohorts are outlined for Part 1 (primary treatment period) in Table 1.

TABLE 1
Objectives and Corresponding Endpoints for Part 1

| | Corresponding Endpoint |
|---|---|
| Primary Objective | |
| To evaluate the | Proportion of participants with mRS score |
| efficacy of | improvement ≥1 from baseline and no use of |
| satralizumab compared | rescue therapy at Week 24 |
| with placebo | |
| on degree of disability | |
| and clinical severity | |
| Secondary Objectives | |
| To evaluate the | Time to mRS score improvement ≥1 from |
| efficacy of | baseline without use of rescue therapy |
| satralizumab compared | Time to rescue therapy |
| with placebo | Time to seizure freedom (seizure freedom |
| | defined as a cessation of seizures for at |
| | least 6 consecutive weeks) or cessation of |
| | status epilepticus without use of rescue therapy |
| | Change in CASE score from baseline at Week 24 |
| | MOCA total score at Week 24 |
| | RAVLT score at Week 24 (LGI1 AIE cohort) |
| | mRS score at Week 24 (as measured on a 7-point |
| | scale; NMDAR AIE cohort) |
| To evaluate the | Incidence, seriousness, and severity of |
| safety of satralizumab | adverse events, with severity determined |
| compared with placebo | according to National Cancer Institute |
| | Common Terminology |
| | Criteria for Adverse Events, Version 5.0 |
| | Change from baseline in targeted vital signs, |
| | clinical laboratory test results, ECG results, |
| | weight, height (<18 years only), and C-SSRS |

ADA=anti-drug antibody; AIE=autoimmune encephalitis; BDI-II=Beck Depression Inventory, second edition; CASE=Clinical Assessment Scale in Autoimmune Encephalitis; C-SSRS=Columbia-Suicide Severity Rating Scale; EEG=electroencephalogram; LGI1=leucine-rich glioma-inactivated 1; MFIS=Modified Fatigue Impact Scale; MOCA=Montreal Overall Cognitive Assessment; mRS=Modified Rankin Scale; NMDAR=N-methyl-D-aspartic acid receptor; PK=pharmacokinetic; QOL=quality of life; RAVLT=Rey Auditory Verbal Learning Test.

### 3. Study Design

### 3.1 Overall Design

### 3.1.1 Overview of Study Design

This Phase III, randomized, double-blind, placebo-controlled, multicenter study is designed to evaluate the efficacy, safety, pharmacokinetics, and pharmacodynamics of satralizumab compared with placebo for the treatment of NMDAR encephalitis and LGI1 encephalitis. For efficacy analyses, the NMDAR AIE and LGI1 AIE cohorts will be treated as separate populations in a basket study design. The study will include a screening period of up to 28 days, during which patients' eligibility will be evaluated for study participation. Screening will be followed by
Part 1: a primary treatment period of 52 weeks
Part 2: an optional extension period lasting approximately 2 years from when the last participant enters the extension period or until commercial satralizumab is available for the treatment of AIE

A pharmacokinetic (PK) interim analysis will be performed to confirm that target concentrations are achieved (see Section 7.4.1).

Approximately 102 participants in the NMDAR AIE cohort and 50 participants in the LGI1 AIE cohort will be enrolled across all sites in a global enrollment phase.

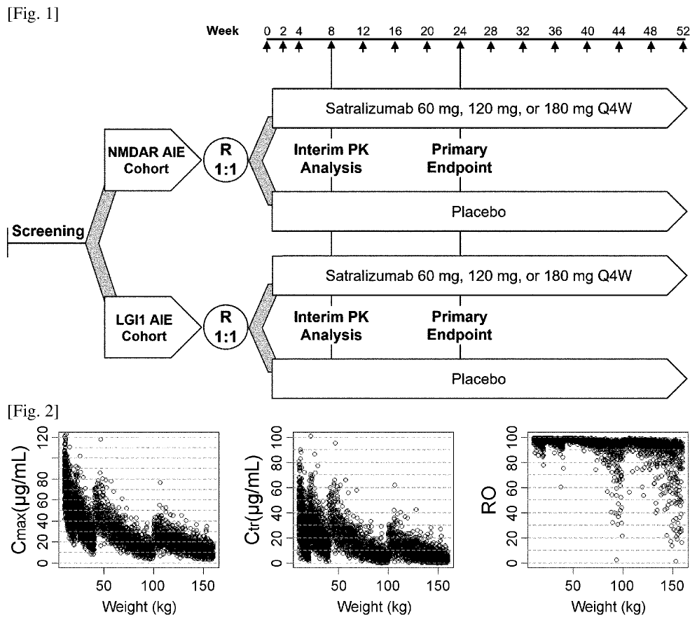

A study schema is provided in FIG. 1.

### 3.1.2 Part 1: Primary Treatment Period

During Part 1, participants will be randomly assigned in a 1:1 ratio to receive placebo or 60 mg (<40 kg), 120 mg (between 40 and 100 kg), or 180 mg (>100 kg) satralizumab in each of the NMDAR AIE and LGI1 AIE cohorts.

Randomization within each cohort will be stratified by:
Patient population: New onset versus incomplete responder (as described in Section 4.1.1)
Region

Blinded study drug will be administered subcutaneously to participants at Weeks 0, 2, 4, and Q4W thereafter until the end of Part 1 as monotherapy or in addition to background treatments.

During Part 1, in addition to satralizumab or placebo, participants in both cohorts may receive background treatment with IST (see Background Treatments, Section 5.1.2) and symptomatic treatments (see Symptomatic Treatments, Section 5.2.2), depending on the stage of disease as outlined in the inclusion criteria.

Participants who receive oral or IV administered corticosteroids at baseline and are not in critical care settings (i.e., intensive therapy unit, high dependency unit) will be tapered off their OCS using a standard taper, starting 4 weeks after randomization (Week 4).

For participants who are in critical care settings (i.e., intensive therapy unit, high dependency units) and receive oral or IV corticosteroids at baseline, steroid tapering will be optional according to physician judgment and, if performed, should follow the tapering schedule for non-critical care settings (shown above). Upon discharge from critical care, the tapering schedule for non-critical care settings will be instituted, providing 4 weeks have elapsed since randomization (Week 4 or later).

Doses of background and symptomatic treatments can be reduced for safety reasons at any time.

A pre-specified interim PK analysis and periodic safety reviews will be performed by an independent Data Monitoring Committee (iDMC) during Part 1. The interim PK analysis will be performed with the purpose of confirming that the achieved satralizumab exposure (and predicted receptor occupancy [RO]) is within the target range. Based on the results from the interim PK analysis and pre-specified criteria, the study drug dose may be increased (see Section 3.3 for the satralizumab dose rationale). For the PK interim analysis, periodic safety reviews and any optional efficacy interim analysis, the Sponsor, participants, and investigators will remain blinded.

### 3.2 Rationale for Study Design

### 3.2.1 Rationale for Study Population and Analysis Groups

The study will investigate satralizumab as treatment in participants with NMDAR and LGI1 encephalitis as separate cohorts within a single trial, with each cohort containing its own placebo arm and analyzed separately with independent Type I error control at a 5% significance level. The population for this study will include participants meeting the clinical criteria for receiving a diagnosis of NMDAR or LGI1 encephalitis (see Section 4.1; adapted from Graus et al. 2016) and whose AIE has led to a decline in function and ability to perform previous activities (defined by a mRS score <2).

The identification of antibodies against neuronal cell surface and synaptic proteins, which are thought to be directly pathogenic, delineates a population with improved response to immunotherapies in general. Among these, the anti-NMDAR- and anti-LGI1-mediated encephalitides represent the most common (Leypoldt et al. 2013) and best characterized syndromes.

NMDAR encephalitis is associated with CSF IgG antibodies against the GluN1 subunit of the NMDAR. These antibodies are highly specific, and their pathogenicity has been demonstrated in cultured neurons and in-vivo models. In clinical practice, antibody studies should include analysis of CSF; a risk of false-negative or false-positive diagnoses exists if only serum is used (Graus et al. 2016).

In clinical practice, access to anti-NMDAR assays, and specifically CSF sampling for anti-NMDAR antibodies, can be inconsistent, delayed, or difficult to achieve. Treatment decisions are often made on the basis of clinical features. As a consequence, criteria for probable NMDAR encephalitis have been published in a consensus document and have shown high sensitivity and specificity in adults (87.2% and 96.7%, respectively; Kaneko 2018), in children in Australia (90% and 96%, respectively; Ho et al. 2017), and in children in Japan (81.2% and 76.9%, respectively; Nishida et al. 2021).

All participants in this study will have received first-line therapy (e.g., high-dose corticosteroids, IVIG, or plasmapheresis) prior to randomization, and participants in the “incomplete responder” category will have received immunotherapeutic treatments beyond 6 weeks after acute first line therapy. The administration of these therapies may influence anti-NMDAR antibody detection, leading to both false-positives and false-negatives (Gruter et al. 2020). Therefore, confirming antibody positivity in a population that has already received treatment may not be a reliable gold standard for diagnosis, and requiring antibody positivity prior to treatment limits applicability and recruitment.

Given that many patients in clinical practice with probable NMDAR encephalitis are treated on the basis of highly suggestive clinical features, and considering the proven utility of the “probable” criteria and possible influence of immunomodulatory therapies on anti-NMDAR antibody detection, the study population will include both definite NMDAR encephalitis (defined by consensus criteria that require CSF antibody positivity and consistent clinical features) and probable NMDAR encephalitis (defined according to consensus criteria that require rigorous clinical characteristics in conjunction with positive paraclinical investigations [CSF and electroencephalogram; EEG]) in the NMDAR population.

Despite concerns with assay interference from prior medication use, serum collection is likely to be more widely adopted globally given that it is more sensitive than CSF in the diagnosis of LGI1 encephalitis (van Sonderen et al. 2016). Although no consensus diagnostic criteria are published, diagnostic criteria requiring key clinical features that are described for limbic encephalitis (the common clinical presentation) in a consensus document (Graus et al 2016) and confirmed in the largest published LGI1 AIE series (van Sonderen et al. 2016) to be used in combination with a positive serum or CSF sample have been developed and are outlined in the protocol.

Due to the unknown significance and pathogenicity of antibodies associated with intracellular antigens and their higher association with cancer, patients with a diagnosis or history of these antibodies will be excluded from the study. Specifically, patients with a known diagnosis or positive historical test of anti-Hu, anti-Ma2, anti-CRMP5, anti-Yo, anti-amphiphysin, AMPA, mGluR5, and GABA^{B} antibodies will be excluded. Similarly, patients with untreated teratoma/thymoma, a history of malignancy (unless deemed cured by adequate treatment with no evidence of recurrence for 5 years before screening), or a confirmed diagnosis of paraneoplastic encephalitis will be excluded from enrollment.

### 3.2.2 Rationale for Control Group

The control group in the study will receive placebo. Participants can continue receiving select background therapy at a stable dose during Part 1, as outlined in Section 5.1.2. The use of placebo in this setting helps to establish the efficacy and safety of satralizumab in clinical situations that represent the real-world scenario for patients with AIE. Placebo-controlled data in the NMDAR and LGI1 encephalitis subtypes will not only provide evidence in these individual syndromes for which there is no approved medication, but may also generate information pertinent to the understanding of the broader range of AIE.

### 3.2.3 Rationale for Pharmacokinetic Sample Collection Schedule

To explore the pharmacokinetics of satralizumab in the NMDAR/LGI1-encephalitis population following longer-term treatment, samples to assess serum concentration of satralizumab will be taken prior to each study drug administration. These assessments will include analysis of the impact of a range of covariates on exposure (e.g., gender, race, age, and bodyweight) and analysis of the relationships between exposure and PD, efficacy, immunogenicity, and safety endpoints to support the recommended dose of satralizumab in the NMDAR/LGI1-encephalitis population. PK assessments will also be used to inform the PK interim analysis to confirm the appropriate doses of satralizumab in the AIE population.

### 3.2.4 Rationale for Immunogenicity Sample Collection

Serum samples for ADAs will be taken in parallel to PK samples to assess the incidence and titer-time profile of ADAs in the NMDAR/LGI1-encephalitis population and the impact on exposure to satralizumab as well as on safety and efficacy. ADA data will be included in the blinded review of PK data at Week 8 to aid the interpretation of the satralizumab concentration data, in addition to the subsequent analysis based on the full study dataset.

### 3.2.5 Rationale for Biomarker Assessments

The study will assess whether biomarkers can aid in characterizing the mechanism of action of satralizumab in NMDAR/LGI1 encephalitis, provide evidence of satralizumab activity in NMDAR/LGI1 encephalitis, or increase the knowledge and understanding of NMDAR/LGI1 encephalitis disease biology.

### 3.2.6 Rationale for Primary Endpoint

The inventors are proposing to use the Modified Rankin Scale (mRS) as the primary outcome measure for this Phase III trial to assess satralizumab benefit on the multiple symptoms impacting the clinical status and disability of participants with either NMDAR or LGI1 encephalitis. The mRS is a score measuring disability and dependency.

The choice of the mRS scale is supported by:
Its proven validity and reliability: The mRS was developed by Dr. John Rankin to assess the degree of stroke severity (7 severity grades: 0=no symptoms at all, to 6=death) and functional limitations in carrying out activities of daily living (i.e., ability to walk, and attend to usual duties and activities) that have been shown to be present and clinically relevant in patients with AIE. The mRS shows good construct validity when correlated with the clinical assessment scale in AIE (CASE), a scale recently developed for AIE which measures symptoms severity (r=0.86, p=0.001; Lim et al. 2019). Test-retest reliability was determined in a sample (n=48) of patients queried in two separate assessments and showed a high degree of reproducibility (r=0.94; Wilson et al. 2005).
Its content validity in NMDAR encephalitis: The complex neuropsychiatric manifestations and global cognitive impairment seen in NMDAR encephalitis lead to disability and dependency in sufferers that is well reflected by the mRS score. Moreover, the score is simple and straightforward for clinicians in acute neurology environments to administer and understand. The mRS has also been used in patients with NMDAR encephalitis to study short- and long-term outcomes of commonly administered medications (Titulaer et al. 2013; Gadoth et al. 2017). Hence, the inventors believe the mRS scale will adequately measure clinically significant neurological improvements in patients with NMDAR encephalitis while representing the most clinically meaningful outcome for a group of patients with disabling, and often diverse, symptomatology (Dubey et al. 2015).
Its content validity in LGI1 encephalitis: Impairment in LGI1 encephalitis, which is dominated by more anatomically restricted cognitive impairment affecting memory and seizures, but also includes behavioral disturbances, has profound effects on abilities and dependency that are amenable to simple assessment and measurement using the mRS score. Additionally, changes in mRS score in patients with positive LGI1 AIE were shown to generally track with changes in key symptoms such as seizure frequency and cognitive deficits (van Sonderen et al. 2016), indicating that the scale is a reasonable measure of how predominant individual symptoms associated with LGI1 encephalitis affect a patient's daily functioning. For these reasons, the mRS has been used in patients with LGI1 AIE to study short- and long-term outcomes in the available literature examining treated natural history (van Sonderen et al. 2016; Gadoth et al. 2017; Gadoth et al. 2018).
Guidance to assess the grades will be tailored to evaluate important aspects of AIE including cognitive deficit (memory loss) during patient interviews. This guidance will be part of the mRS mandatory training across all sites to ensure consistency.

Finally, the mRS has also been utilized as the primary endpoint in several randomized controlled and open-label studies in AIE, including patients with both NMDAR and LGI encephalitis (NCT04372615, NCT03993262, NCT03542279, and NCT04175522). Available data on the mRS scale in AIE have been used to help inform the proposed study design, in particular regarding expected changes in the placebo arm (Gadoth et al. 2017; Titulaer et al. 2013).

The primary endpoint in the NMDAR AIE and LGI1 AIE cohorts will assess the difference between satralizumab and placebo in the proportion of responders at Week 24 in participants with NMDAR encephalitis and LGI1 encephalitis, respectively.

A responder is defined as a participant with a mRS score improvement from baseline of 1 point or greater and no use of rescue therapy at Week 24. A change of one mRS grade is considered to be clinically significant based on the range of severity covered by the scale grade (Banks and Marotta 2007; Harrison et al. 2013) and is used in AIE clinical practice and observational studies to represent a meaningful change in the patient's capacity to engage in activities of daily living. The inventors propose to include patients with at least an inability to carry out all their previous activities (as measured by mRS of 2 or more) at baseline. Therefore, a score improvement of 1 (rather than mRS score 0-2, that generally corresponds to a “good outcome” on the scale) would adequately capture meaningful improvements in all participants in the study with differing levels of symptomatology and degree of presenting disability. Additional data using the mRS scale will be collected as part of the secondary and exploratory endpoints (mRS score [as measured on a 7-point scale] and proportion of participants with mRS score of 2 or less).

### 3.3 Justification for Dose and Schedule

Weight-tiered dosing via SC injection will be used in this study for the investigation of efficacy and safety of satralizumab in NMDAR and LGI1 encephalitis as shown in Table 2.

TABLE 2
Dosing Regimen for Investigation in Phase III Study of Satralizumab
for the Treatment of NMDAR and LGI1 Encephalitis

| | |
|---|---|
| Body Weight | |
| At Baseline | Dose and Regimen |

| | | |
|---|---|---|
| <40 | kg | 60 mg administered at Weeks 0, 2, 4, |
| | | and Q4W thereafter as a SC injection |
| ≥40-100 | kg | 120 mg administered at Weeks 0, 2, 4, |
| | | and Q4W thereafter as a SC injection |
| >100 | kg | 180 mg administered at Weeks 0, 2, 4, |
| | | and Q4W thereafter as a SC injection |

PK = pharmacokinetic; Q4W = every 4 weeks.
Notes:
Doses may be increased following the PK interim analysis, see details below.

The dosing regimen is based on a combination of sources of information, including PK, PD, and safety data for satralizumab for the initial development in NMOSD.

The 120-mg fixed dosing regimen (administered at Weeks 0, 2, and 4, and Q4W thereafter) investigated in the Phase III studies in NMOSD was associated with high predicted trough-receptor occupancy at steady state (RO_{tr,ss}) in most participants (median value of 95% or more) and was shown to be safe and efficacious in all body weight groups (i.e., 40 kg and above). The 4-week maintenance dosing interval is supported by the half-life for satralizumab of approximately 30 days. The few NMOSD participants with predicted RO_{tr,ss} values of less than 80% generally had baseline body weights >100 kg, and though the safety profile was similar across body weight groups, the satralizumab exposures in the lowest body weight participants were in excess of those required to maintain near-maximal RO_{tr,ss}.

Given the anticipated similar target expression between participants with NMOSD and NMDAR/LGI1 encephalitis, exposure similar to that seen in NMOSD is expected to also be effective in NMDAR and LGI1 encephalitis. Therefore, the inventors have performed simulations using the existing popPK model (based on data in healthy volunteers and in participants with NMOSD) to estimate the dose required to achieve the same near-maximal RO_{tr,ss} values for participants with NMDAR and LGI1 encephalitis across the expected body weight range, maximizing the potential for efficacy.

These simulations indicated that the proposed body weight-tiered dose regimens would be expected to achieve similar median maximum concentration observed (Cₘₐₓ) at steady state (C_{max,ss}) values and trough concentration at steady state (C_{tr,ss}) values to those observed in the NMOSD Phase III studies, which were associated with near-maximal RO throughout the dose interval (see FIG. 2). The range of predicted exposures in participants weighing >100 kg receiving 180 mg satralizumab does not significantly exceed the maximum exposures achieved in the Phase III trials in NMOSD, and therefore remains within the existing exposure-safety coverage. Conversely, the predicted exposures for the 60-mg regimen in participants <40 kg are expected to be sufficient to maintain high target engagement over the dose interval and avoid unnecessary overexposure.

The assumption in setting initial doses for this Phase III study is that the pharmacokinetics of satralizumab in participants with NMDAR and LGI1 encephalitis is similar to those with NMOSD. However, the inventors are mindful that PK differences are possible between populations, as seen in higher clearance (CL) values in healthy volunteers compared with the NMOSD population (covariate value: 95.8%; 95% CI: 67.5 to 124.1). Therefore, the proposed study design makes provision for a PK interim analysis to either confirm the initial doses, or, if the CL is higher in the NMDAR/LGI1 population (as observed in healthy volunteers), to increase the doses to pre-specified levels on the recommendation of the iDMC, with the Sponsor remaining blinded to help maintain the integrity of this pivotal study. Further detail on the proposed PK interim analysis can be found in Section 7.4.1.

Simulation using the existing popPK model has been used to define alternative doses in the case that the doses initially proposed do not achieve target exposures. The use of the existing RO model as the basis for prediction of RO in this interim analysis is considered appropriate, given that the target is the same (i.e., for NMOSD and NMDAR/LGI1 encephalitis) and similar target expression is expected for NMDAR and LGI1 encephalitis. In the case that CL values in this study are reflective of those in healthy volunteers (higher than those in the NMOSD population), the dose adaptation option will be to increase the dose to the pre-defined dosing regimen of 120 mg (<40 kg), 180 mg (between 40 and 100 kg), and 240 mg (>100 kg) for participants. In either case, the chosen dosing regimen will be associated with exposures that do not significantly exceed the existing exposure-safety coverage.

PK parameters in adolescent participants with NMOSD were similar to those in adult participants, and the predicted exposures resulting from this dosing regimen are supported by the existing safety profile established in the Phase III studies in adult and adolescent participants with NMOSD.

### 3.4 End of Study Definition

A participant is considered to have completed the study if he or she has completed all phases of the study, including the last visit/last scheduled procedure.

The end of this study is defined as the date of the last visit of the last participant in the study across both cohorts or the date at which the last data point required for statistical analysis or SFU is received from the last participant in either cohort, whichever occurs later.

4. The End of Part 1 is Expected to Occur 52 Weeks after the Last Participant is Enrolled Study Population

Participants with NMDAR encephalitis and participants with LGI1 encephalitis will be enrolled during the global enrollment phase of this study.

Prospective approval of protocol deviations to recruitment and enrollment criteria, also known as protocol waivers or exemptions, is not permitted.

### 4.1 Inclusion Criteria

Participants in the NMDAR AIE cohort must meet the inclusion criteria outlined in Sections 4.1.1 and 4.1.2. Participants in the LGI1 AIE cohort must meet the inclusion criteria outlined in Sections 4.1.1 and 4.1.3.

### 4.1.1 Inclusion Criteria for All Participants

Participants are eligible to be included in the study only if the following criteria apply:
Capable of giving signed informed consent, which includes compliance with the requirements and restrictions listed in the Informed Consent Form and in this protocol
Reasonable exclusion of tumor or malignancy before baseline visit (randomization)
Onset of AIE symptoms within 9 months before randomization
Meet the definition of “New Onset” or “Incomplete Responder” AIE Participants will be sub-classified as either “New Onset” or “Incomplete Responder” based on the interval between their initial first line treatment and the start of study therapy, as well as additional treatments received, as follows:
New Onset: defined as a participant with NMDAR or LGI1 AIE who satisfies the following criteria:
Has a stable (for at least 24 hours) mRS score of 2 or more, measured at baseline
Has received their first acute first-line therapy within 6 weeks prior to randomization (baseline visit)

Acute first-line therapy is defined as a minimum of 3 days of IV methylprednisolone at a dosage of 500 mg or more per day (or equivalent oral glucocorticoid dose) and/or at least 3 days of IVIG therapy and/or PLEX, or any combination of these. Both OCS and/or repeated courses of acute first-line therapy (following the initial course) are permitted. For participants with LGI AIE receiving OCS, a dose of 20 mg or more prednisolone (or its equivalent) daily at randomization is required. After randomization, the taper schedule in Section 3.1.2 should be followed.
Has not received prior treatment with rituximab or any other ISTs (e.g., cyclophosphamide, mycophenolate mofetil, methotrexate, cyclosporine, tacrolimus, azathioprine) or tocilizumab for AIE
Incomplete Responder: defined as a participant with NMDAR or LGI1 AIE who satisfies the following criteria:
Has a stable (for at least 24 hours) mRS score of 2 or more, measured at baseline
Has received their first acute first-line therapy more than 6 weeks prior to randomization (baseline visit). Acute first-line therapy is defined as a minimum of 3 days of IV methylprednisolone at a dosage of 500 mg or more per day (or equivalent oral glucocorticoid dose) and/or at least 3 days of IVIG therapy and/or PLEX, or any combination of these.
Has received immunotherapy beyond their first acute first-line therapy course. The following criteria must be satisfied:
For participants who have received rituximab, a therapeutic course initiated at least 2 months prior to screening, the last dose administered at least 4 weeks prior to randomization, and no improvement in mRS score within 4 weeks prior to randomization
For participants receiving other ISTs (i.e., mycophenolate mofetil, cyclophosphamide [IV only], or azathioprine), treatment for at least 2 months prior to screening, at a dose that has been stable for at least 4 weeks prior to screening and with no improvement in mRS score within the 4 weeks prior to randomization
For participants receiving OCS, a stable dose of 20 mg or more prednisolone (or its equivalent) daily, and no increase of steroid dose during the 4 weeks prior to screening, and no improvement in mRS score within the 4 weeks prior to randomization
For participants that have received repeated courses (pulses) of acute first-line therapy, treatment must have been completed at least 2 weeks before randomization (baseline visit)

### 4.1.2 Additional Inclusion Criteria for the NMDAR AIE Cohort

In addition to the criteria outlined in Section 4.1.1, participants are eligible to be included in the NMDAR AIE cohort if the following criteria apply:
Age of 12 years or older at the time of signing Informed Consent Form For adolescent participants and participants otherwise unable to give consent due to the severity of their disease, the Informed Consent Form may be signed by a legally authorized representative and participant assent obtained, as per local requirements.
Signed Assent Form when appropriate, as determined by patient's age and individual site and country standards
Diagnosis of probable or definite NMDAR encephalitis, as follows: Diagnosis of probable NMDAR encephalitis can be made when the following three criteria have been met:
1. Rapid onset (less than 3 months) of at least four of the six following major groups of symptoms:
   Abnormal (psychiatric) behavior or cognitive dysfunction
   Speech dysfunction (pressured speech, verbal reduction, mutism)
   Seizures
   Movement disorder, dyskinesias, or rigidity/abnormal postures
   Decreased level of consciousness
   Autonomic dysfunction or central hypoventilation
2. At least one of the following laboratory results:
   Abnormal EEG (focal or diffuse slow or disorganized activity, epileptic activity, or extreme delta brush)
   CSF with pleocytosis or oligoclonal bands
3. Reasonable exclusion of alternative causes and other well-defined syndromes of encephalitis (e.g., Bickerstaff's brainstem encephalitis, acute disseminated encephalomyelitis, Hashimoto's encephalopathy, primary angiitis of the central nervous system [CNS], Rasmussen's encephalitis)
A diagnosis of probable NMDAR encephalitis can also be made in the presence of three of the above groups of symptoms in criteria 1 if accompanied by a systemic teratoma.

Diagnosis of definite NMDAR encephalitis can be made when three of the following criteria have been met:
1. The presence of one or more of the six major groups of symptoms stated in criteria 1 for probable NMDAR encephalitis
2. Documented history or presence at screening of anti-NMDAR (GluN1) IgG antibodies detected in the CSF using a cell-based assay
3. Reasonable exclusion of alternative causes and other well-defined syndromes of encephalitis (e.g., Bickerstaff's brainstem encephalitis, acute disseminated encephalomyelitis, Hashimoto's encephalopathy, primary angiitis of the CNS, Rasmussen's encephalitis)

### 4.1.3 Additional Inclusion Criteria for the LGI1 AIE Cohort

In addition to the criteria outlined in Section 4.1.1, participants are eligible to be included in the LGI1 AIE cohort if the following criteria apply:
Age of 18 years or older at the time of signing Informed Consent Form

For participants otherwise unable to give consent due to the severity of their disease, the Informed Consent Form may be signed by a legally authorized representative and participant assent obtained, as per local requirements.
Diagnosis of LGI1 encephalitis

Diagnosis of LGI1 encephalitis can be made in the presence of the following criteria:
1. Documented history or presence at screening of anti-LGI1 IgG antibodies (in serum or CSF) using a cell based assay
2. Subacute onset (progression of less than 4 months) of working memory deficits, seizures (including facio-brachio dystonic seizures), or psychiatric symptoms suggesting involvement of the limbic system

Diagnosis of LGI1 encephalitis should reasonably exclude alternative causes and other well-defined syndromes of encephalitis (e.g., Bickerstaff's brainstem encephalitis, acute disseminated encephalomyelitis, Hashimoto's encephalopathy, primary angiitis of the CNS, Rasmussen's encephalitis)

### 4.2 Exclusion Criteria

Participants are excluded from the study if any of the following criteria apply:
Any untreated teratoma or thymoma at baseline visit (randomization)

Teratoma or thymoma detected prior to or during the screening period is allowed if deemed cured after treatment (usually surgical removal) by 1 week prior to baseline
History of carcinoma or malignancy, unless deemed cured by adequate treatment with no evidence of recurrence for at least 5 years before screening (except basal cell and squamous cell carcinomas of the skin, or in situ carcinoma of the cervix uteri that have been completely excised and cured)
For patients with NMDAR AIE, history of negative anti-NMDAR antibody in CSF using a cell based assay within 9 months of symptom onset
Historically known positivity to an intracellular antigen with high cancer association (e.g., anti-Hu, anti-Ma2, anti-CRMP5, anti-Yo, anti-amphiphysin, AMPA, mGluR5, and GABA^{B}) or GAD-65
Historically known positivity to any cell surface neuronal antibodies other than NMDAR and LGI1 (e.g., caspr2, IgLON5, DPPX, GABA^{A}, and neurexin-3 alpha)
Confirmed paraneoplastic encephalitis
Confirmed central or peripheral nervous system demyelinating disease (e.g., multiple sclerosis, chronic inflammatory demyelinating polyneuropathy)
Alternative causes of associated symptoms, including CNS infections, septic encephalopathy, metabolic encephalopathy, epileptic disorders, mitochondrial disease, Klein-Levin syndrome, Creutzfeldt-Jakob disease, rheumatologic disorders, Reyes syndrome, or inborn errors of metabolism
History of herpes simplex virus encephalitis in the previous 24 weeks

### 5. Study Treatment(s) and Concomitant Therapy

Study treatment is defined as any investigational treatment, marketed product, placebo, or medical device intended to be administered to a study participant according to the study protocol.

The investigational medicinal products (IMPs) for this study are satralizumab and placebo. Background therapy, rescue therapy, and symptomatic treatments are considered non-IMPs.

### 5.1 Study Treatments Administered

### 5.1.1 Satralizumab and Placebo

The test product in this study is satralizumab. In this protocol, “study drug” refers to satralizumab or placebo (assigned in addition to background therapy). In Part 1, study drug will be administered at a site visit at Weeks 0, 2, 4, and Q4W thereafter. Participants will receive satralizumab according to body weight at 60 mg (<40 kg), 120 mg (between 40 and 100 kg), or 180 mg (>100 kg).

Study drug will be administered by SC injection in the abdominal or femoral region after all other study-related procedures have been performed at a site visit.

### 5.1.2 Background Treatments

Participants classified as Incomplete Responders (see Section 4.1.1) may have received or will continue to receive background treatment(s) in addition to study drug during Part 1.

Background treatment medications are exemplified below:
Azathioprine
Mycophenolate mofetil
IV Cyclophosphamide

For symptomatic medications permitted during the study, see Section 5.2.2.

For participants who discontinue background treatment prior to study entry, study eligibility for full blood counts and differential blood counts (see Section 4.2) must be met at randomization.

5.2 concomitant therapy

Concomitant therapy consists of any medication (e.g., prescription drugs, over-the-counter drugs, vaccines, herbal or homeopathic remedies, and/or nutritional supplements) used by a participant in addition to protocol-mandated treatment from 7 days prior to initiation of study treatment to the final SFU visit. All such medications must be recorded on the Concomitant Medications eCRF along with the following information:
Reason for use
Dates of administration, including start and end dates
Dosage information, including dose and frequency

The Medical Monitor may be consulted if there are any questions related to concomitant or prior therapy.

Paracetamol/acetaminophen, at doses of 2 g or less per day is permitted for use at any time during the study and during the screening period.

### 5.2.1 Rescue Therapy

Rescue therapy is defined as the initiation or increase in dose of an IST medication (see Section 5.1.2 for details on permitted background therapies), rituximab, OCS, the use of repeat first-line immunotherapy (IVIG, IV methylprednisolone, or plasmapheresis), or the failure to taper OCS according to the protocol-directed taper.

The following rescue procedure may be used:
Plasma Exchange (PLEX)

The following rescue medications may be used:
IVIG
Rituximab
IV Cyclophosphamide
Azathioprine
Mycophenolate mofetil
Corticosteroids (OCS or IV methylprednisolone)

### 5.2.2 Symptomatic Treatments

Use of the following medications commonly used for symptomatic management of AIE (Abboud et al. 2021) is permitted, as described below:
AEDs (e.g., carbamazepine, lacosamide, levetiracetam)
Pain medications (e.g., topical agents, anti-epileptic drugs [e.g., pregabalin, gabapentin, carbamazepine])
Antidepressants (e.g., tricyclic antidepressants, serotonin norepinephrine reuptake inhibitors [e.g., duloxetine])
Antipsychotic agents (e.g., quetiapine)
Mood stabilizers (e.g., valproic acid)
Beta-blockers (e.g., propranolol)
Alpha-2 blockers (e.g., clonidine)
Alpha-adrenergic agonists (e.g. midodrine)
Acetylcholine esterase inhibitors (e.g., pyridostigmine) for increased sympathetic drive
Non-opioid analgesics (e.g., paracetamol or acetaminophen, non-steroidal anti-inflammatory drugs)
Modafinil or melatonin for sleep disorder
Antimuscarinics, muscle relaxants, antidopaminergics, and dopamine agonists for movement disorders

### 5.2.3 Other Permitted Therapy

In general, investigators may manage a participant's care (including preexisting conditions) through use of supportive therapies, as clinically indicated and per local standard practice, with the exception of prohibited therapies and taking into account cautionary therapies. Participants who experience infusion-associated symptoms may be treated symptomatically with paracetamol/acetaminophen, ibuprofen, diphenhydramine, and/or H₂-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and beta-2-adrenergic agonists). Premedication with antihistamines, antipyretic medications, and/or analgesics may be administered at the discretion of the investigator.

Use of the following concomitant therapies is permitted as described below:
Oral contraceptives (see Section 4.1)
Hormone-replacement therapy
Background treatments as listed in Section 5.1.2.
Treatment with topical (e.g., ophthalmic, nasal, otic, cutaneous) corticosteroids for adverse events (i.e., indications other than background AIE therapy), provided that the duration of treatment is kept as short as possible
Non-medication interventions (e.g., individual psychotherapy, cognitive behavioral therapy, physical therapy and rehabilitative therapy)

### 6. Study Assessments and Procedures

### 6.1 Efficacy Assessments

Participants will undergo efficacy assessments at screening and at timepoints thereafter. Assessments to evaluate efficacy will include participant-reported (PRO), performance-based (PerfO) and clinician-reported outcomes (ClinRO) (see Section 6.2); and any optional assessments that capture disease severity (including lumbar puncture). Additionally, EEG will also be analyzed as an exploratory outcome (see Section 6.3).

### 6.2 Clinical Outcome Assessments

Performance-based outcome (PerfO), and clinician-reported outcome (ClinRO) instruments will be completed to assess the treatment benefit of satralizumab.

Clinical outcome assessment instruments are listed in Table 3.

TABLE 3
Clinical Outcome Assessments

| Data Type | Instrument | Description |
|---|---|---|
| PerfO | MOCA | 6.2.2.3 |
| | RAVLT | 6.2.2.4 |
| ClinRO | mRS | 6.2.2.1 |
| | CASE | 6.2.2.2 |
| | C-SSRS | 6.2.2.5 |
| Diary | Seizure Diary | 6.2.2.6 |

CASE = Clinical Assessment Scale of Encephalitis; ClinRO = clinician-reported outcome, C-SSRS = Columbia-Suicide Severity Rating Scale; MOCA = Montreal Cognitive Assessment; mRS = Modified Rankin Scale; PerfO = performance-based outcome; RAVLT = Rey Auditory Verbal Learning Test.

### 6.2.1 Data Collection Methods for Clinical Outcome Assessments

At the clinic, instruments will be administered before the participant receives any information on disease status, prior to the performance of non-PRO (participant-reported outcome) assessments, and prior to the administration of study treatment, unless otherwise specified.

PerfO and ClinRO instruments will be completed at the clinic at specified timepoints during the study. PerfO and ClinRO instruments will be administered prior to completion of the physical examination, prior to any safety assessments, and prior to the administration of study treatment. Clinicians must complete the official version of each ClinRO instrument, as provided by the Sponsor. Instruments must not be copied from the protocol.

### 6.2.2 Description of Clinical Outcome Assessment Instruments

6.2.2.1 Modified Rankin Scale (mRS)

The mRS is a scale developed to measure global disability after a stroke and has been widely applied to evaluate primary outcomes in randomized control trials of acute stroke management (Rankin 1957). The 7-point scale is weighted toward motor function and evaluates the ability to walk to measure functional independence. The mRS is a clinician-rated assessment and consists of six grades from 0 to 5, with 0 corresponding to no symptoms and 5 corresponding to severe disability. Higher scores denote more severe disability. A separate category (of 6) is usually added to classify patients who die before the assessment window. An example of the scale is shown by Banks J L et al. (Stroke. 2007; 38:1091-1096). The mRS is usually used to measure neurological outcomes in AIE in the absence of a validated disease-specific scale. The mRS and its associated structure interview takes approximately 15 minutes to complete and will be administered at the specified timepoints.

### 6.2.2.2 Clinical Assessment Scale of Encephalitis (CASE)

The CASE is a novel scale for rating severity in patients with diverse AIE syndromes and consists of nine items (seizure, memory dysfunction, psychiatric symptoms, consciousness, language problems, dyskinesia/dystonia, gait instability and ataxia, brainstem dysfunction, and weakness). Each item is assigned a value of up to three points, with higher scores corresponding to greater severity of symptoms. The total score ranges from 0 to 27. The CASE has been developed for application in clinical practice and might help overcome the limitations of current outcome scales for AIE in a clinical trial context in the future (Lim et al. 2019). An example of the scale is shown by Lim J A et al. (Ann Neurol. 2019 March; 85(3):352-358.). The CASE takes approximately 10 minutes to complete and will be administered at the specified timepoints.

### 6.2.2.3 Montreal Cognitive Assessment (MOCA)

The MOCA is a 30-item measure of global cognitive function developed to detect early suspected cognitive deficits. It covers the following cognitive domains:
Orientation: knowing the day, date, and your present location
Short-term memory recall: ability to hear a word and repeat it back a short time later
Focus and spatial awareness: connecting numbered dots in sequence and drawing 3-dimensional shapes
Language: ability to speak and understand whole sentences and remember the names of well-known animals or objects
Concentration: repeating simple sequences forward and backward
Visuospatial: performing the Clock Drawing Test (i.e., for evaluating dementia warning signs)

The MOCA takes only a few minutes to perform and is available in three versions to decrease possible learning effects when it is administered every 3 months or less. MOCA scores range between 0 and 30. Higher scores denote better cognitive function. A score of 26 or over is interpreted as normal cognitive functioning (see https://www.mocatest.org/). The MOCA will be administered at the specified timepoints.

### 6.2.2.4 Rey Auditory Verbal Learning Test (RAVLT)

The RAVLT is a neuropsychological test using language to assess immediate memory span, new learning, susceptibility to interference, and recognition memory. The number of words recalled on each trial is the measure of performance.

After five repetitions of free-recall, a second “interference” list (List B) is presented in the same manner and the participant is asked to recall as many words from List B as possible. After the interference trial, the participant is immediately asked to recall the words from List A, which they had heard five times previously. After a 30-minute delay, the participant is asked to again recall the words from List A. After this “delayed recall” task, a list of 50 words is presented containing all of the words from Lists A and B, in addition to 20 phonemically and/or semantically similar words. This trial directly tests recognition memory, as opposed to free-recall. The multiple memory processes assessed by the RAVLT provide rich data regarding memory abilities (Peaker and Stewart 1989; Spreen and Strauss 1991). Higher scores denote better memory abilities. The RAVLT takes approximately 35 minutes to complete and will be administered at the specified timepoints.

### 6.2.2.5 Columbia-Suicide Severity Rating Scale (C-SSRS)

The C-SSRS is a clinician-rated tool used to assess the lifetime suicidality of a patient and to track suicidal events throughout treatment. The scale will be administered at the specified timepoints for prompt recollection of suicidal ideation, including the intensity of the ideation, behavior, and attempts with actual and potential lethality. The “C-SSRS at baseline” will be collected at baseline, and the “C-SSRS since last visit” will be collected at subsequent visits. The C-SSRS takes 5-10 minutes to complete. Examples of the C-SSRS forms are available online at: https:/cssrs.columbia.edu/.

### 6.2.2.6 Seizure Diary

A seizure diary will be provided for completion by the participant or caregiver. Instructions will be provided on how to collect seizure type, frequency, and duration.

### 6.3 Additional Exploratory Assessments

### 6.3.1 Electroencephalographies

EEGs will be conducted on participants at specified timepoints.

Details of the EEG assessment and data handling will be described in a separate EEG recording and handling manual.

### 7. Statistical Considerations

This study will compare satralizumab with matching placebo, administered for 52 weeks, in participants with NMDAR and LGI1 encephalitis as defined by the study eligibility criteria. The NMDAR AIE and LGI1 AIE cohorts will be treated as separate populations and analyzed separately. Each cohort will have independent type I error control at a 5% significance level. Unless specifically stated otherwise, the statistical considerations are the same for the NMDAR AIE and LGI1 AIE cohorts. Participants receiving background therapy will be required to follow protocol-defined rules for Part 1, as outlined in Section 5.1.2.

### 7.1 Statistical Hypotheses

This study aims to demonstrate the superiority of satralizumab over placebo. The primary and secondary efficacy analyses will compare satralizumab with placebo in each of the following cohorts:
NMDAR AIE cohort: adults and adolescents with definite or probable NMDAR encephalitis
LGI1 AIE cohort: adults with LGI1 encephalitis

The primary efficacy analysis will compare satralizumab with placebo at Week 24. The following null and alternative hypotheses will be tested at a two-sided significance level of 5% in each of the NMDAR and LGI1 AIE cohorts:

$${H}_{0} : {p}_{satralizumab} = {p}_{placebo} versus {H}_{1} : {p}_{satralizumab} \neq {p}_{placebo} ,$$

for which p_{satralizumab} and p_{placebo} refer to the proportion of participants with at least 1-point improvement from baseline in mRS and no use of rescue therapy at Week 24 in the satralizumab and placebo groups, respectively.

Null and alternative hypotheses of a similar form will be tested for all secondary efficacy analyses. Note that all hypothesis tests will be two-sided unless stated otherwise.

### 7.2 Sample Size Determination

In the NMDAR ATE cohort, approximately 102 participants with NMDAR encephalitis will be randomly assigned to study treatment such that approximately 92 evaluable participants complete the study in this cohort. In the LGI1 AIE cohort, approximately 50 participants with LGI1 encephalitis will be randomly assigned to study treatment such that approximately 45 evaluable participants complete the study in this cohort.

For each cohort, participants will be randomized in a 1:1 ratio to each treatment group (satralizumab or placebo). Randomization will be stratified by the following criteria:
Patient population: new onset versus incomplete responders (as described in Section 4.1.1)
Region

The estimated sample size required to demonstrate efficacy with regard to the mRS was calculated separately for the NMDAR AIE and LGI1 AIE cohorts.

Based on these assumptions and using a two-sided significance level of 5%, the sample size to achieve 80% power in each cohort was estimated at 102 participants (51 per group) in the NMDAR AIE cohort and 50 participants (25 per group) in the LGI1 AIE cohort.

### 7.3 Statistical Analyses

The Statistical Analysis Plan (SAP) will be finalized prior to study unblinding and database lock, and it will include a more technical and detailed description of the statistical analyses described in this section. This section is a summary of the planned statistical analyses of the most important endpoints, including primary and key secondary endpoints.

### 7.3.1 Primary Endpoint

### 7.3.1.1 Primary Efficacy Estimand and Estimator

The primary efficacy objective is to evaluate the efficacy of satralizumab versus placebo on degree of disability and clinical severity in participants with NMDAR and LGI1 encephalitis. The primary comparison of interest is the difference between the placebo and satralizumab groups in the proportion of participants with an improvement from baseline in mRS of at least 1 point and no use of rescue therapy at Week 24. The primary efficacy estimand will be evaluated separately in the NMDAR and LGI1 AIE cohorts.

If a participant withdraws from treatment, the reason for withdrawal will be classified as either study drug or condition related (SDCR) or not study drug or condition related (NSDCR). More details of this classification will be given in the SAP. The primary comparison will include both participants who withdraw due to SDCR and NSDCR reasons, with the assumption that participants who withdrew due to NSDCR reasons would have continued receiving their randomized treatment.

The primary comparison will be made regardless of whether a participant has a treatment interruption (e.g., due to infection) or a change in background therapy not considered rescue.

The elements of the primary estimand, evaluating the proportion of participants with a 1 point or more improvement from baseline in mRS and no use of rescue therapy at Week 24, regardless of SDCR withdrawal from study treatment, treatment interruption or change in background therapy not considered rescue, and assuming participants with NSDCR withdrawals would have continued to receive their randomized treatment, are defined in Table 4. The primary analysis approach and determination of sample size is aligned with the primary estimand.

TABLE 4
Attributes of Primary Estimand

| | |
|---|---|
| Population | Participants with NMDAR or LGI1 encephalitis, |
| | as defined in Section 4.1 |
| Variable or endpoint | A ≥1-point improvement from baseline in mRS |
| | and no use of rescue therapy at Week 24 |
| Treatment | Satralizumab versus matching placebo, |
| | administered for 52 weeks, in combination with |
| | background therapy |
| Intercurrent events | |
| SDCR withdrawal | Treatment policy ^{a} |
| from study treatment | |
| NSDCR withdrawal | Hypothetical ^{b} |
| from study treatment | |
| Taking rescue therapy | Composite ^{c} (included in variable definition) |
| Treatment interruption | Treatment policy ^{a} |
| (e.g., due to infection) | |
| Change in background | Treatment policy ^{a} |
| therapy not | |
| considered rescue | |
| Summary measure | Odds ratio at Week 24 |
| Method of estimation | The hypothesis will be tested using a Cochran- |
| | Mantel-Haenszel test stratified by the |
| | randomization stratification factors. An estimate |
| | of the treatment effect (odds ratio) will be |
| | presented with associated 95% CI calculated |
| | using a Wald normal approximation. |

LGI1 = leucine-rich glioma inactivated 1; mRS = Modified Rankin Scale; NMDAR = N-methyl-D-aspartic acid receptor; NSDCR = not study drug or condition related; SDCR = study drug or condition related.
^{a} Treatment policy: The value for the variable is used regardless of whether or not the intercurrent event occurs.
^{b} Hypothetical: A scenario is envisioned in which the intercurrent event would not occur.
^{c} Composite: Incorporation of intercurrent events in the definition of the endpoint.

Supplementary estimands incorporating different approaches for handling intercurrent events will be detailed in the SAP and will include using a treatment policy approach for the intercurrent event of taking rescue therapy.

### 7.3.1.2 Missing Data Handling

In the case of missing data, longitudinal mRS data will be imputed and then the responder definition (mRS improvement of at least 1 point) will be applied. For NSDCR withdrawals, data will be censored at the time of the treatment withdrawal. Any data collected after NSDCR withdrawal will be disregarded. Missing data (including use of rescue therapy) following a withdrawal that is determined to be NSDCR will be imputed using multiple imputation with a “Missing at Random” assumption. Missing data following a withdrawal that is determined to be SDCR will be imputed using reference based multiple imputation with a “Copy Reference” assumption. Use of rescue therapy will be imputed based on the placebo arm. This approach will only be used if observed data following a treatment withdrawal are not available. Where observed data are available, they will continue to be included in the analysis. Data following treatment interruption (e.g., due to infection) and a change in background therapy not considered rescue will be included in the analysis. The robustness of the primary estimation method will be explored by a series of sensitivity estimators based on varying assumptions underlying the multiple imputation strategy and classification of treatment withdrawal intercurrent events as SDCR or NSDCR.

Additional details will be provided in the SAP.

### 7.3.2 Secondary Endpoints

The first endpoint to be tested in both cohorts will be the primary endpoint. Subsequently, analysis of the secondary endpoints will follow a cohort specific hierarchy.

The order of the secondary endpoints presented below does not necessarily reflect the hierarchy for each cohort.

The following secondary efficacy endpoints will be analyzed using the time to event estimand and estimator:
Time to mRS score improvement of at least 1-point from baseline without use of rescue therapy
Time to rescue therapy
Time to seizure freedom (seizure freedom defined as a cessation of seizures for at least 6 consecutive weeks) or cessation of status epilepticus without use of rescue therapy

The following secondary efficacy endpoint will be analyzed using the mean change estimand and estimator:
Change in CASE score from baseline at Week 24

The following secondary efficacy endpoints will be analyzed using the mean score estimand and estimator:
MOCA total score at Week 24
RAVLT score at Week 24 (LGI1 AIE cohort)

The following secondary efficacy endpoints will be analyzed using the categorical score estimand and estimator:
mRS score at Week 24 (as measured on a 7-point scale; NMDAR AIE cohort) Supplementary estimands for each endpoint will be detailed in the SAP.

7.3.2.1 Time to Event Estimand and Estimator The elements of the time to event estimand are defined in Table 5 using the time to rescue therapy endpoint as an example. The comparison of interest is the difference between the placebo and satralizumab groups in the time to rescue therapy, regardless of SDCR withdrawal from study treatment, treatment interruption or change in background therapy not considered rescue, and assuming participants with NSDCR withdrawals would have continued to receive their randomized treatment.

TABLE 5
Attributes of Time to Event Estimand

| | |
|---|---|
| Population | Participants with NMDAR or LGI1 encephalitis, as defined |
| | in Section 4.1 |
| Variable or endpoint | Time to rescue therapy |
| Treatment | Satralizumab versus matching placebo, administered for |
| | 52 weeks, in combination with background therapy |
| Intercurrent events | |
| SDCR withdrawal from study | Treatment policy ^{a} |
| treatment | |
| NSDCR withdrawal from study | Hypothetical ^{b} |
| treatment | |
| Death | Composite ^{c} (although not explicitly stated, treated as an |
| | event) |
| Treatment interruption (e.g., due | Treatment policy ^{a} |
| to infection) | |
| Change in background therapy | Treatment policy ^{a} |
| not considered rescue | |
| Summary measure | Hazard ratio |
| Method of estimation | To account for the competing event of death before |
| | observation of rescue therapy, probability estimates will |
| | be based on the cumulative incidence function. This will |
| | be estimated using the non-parametric Aalen-Johansen |
| | estimator. To compare treatment groups, the relative risk |
| | will be calculated based on these estimated probabilities. |
| | To assess the effect of treatment on estimated |
| | probabilities over the entire time span, subdistribution |
| | hazards corresponding to the cumulative incidence |
| | functions will be compared using the Gray test. Similar |
| | methods will be implemented for all time to event |
| | endpoints although the competing events may differ. |
| | Participants who withdraw from study treatment due to |
| | NSDCR reasons will be censored at the date of treatment |
| | withdrawal. Missing data following a withdrawal that is |
| | determined to be SDCR will be imputed as described for |
| | the primary endpoint (Section 7.3.1.2). |

LGI1 = leucine-rich glioma inactivated 1; NMDAR = N-methyl-D-aspartic acid receptor; NSDCR = not study drug or condition related; SDCR = study drug or condition related.
^{a} Treatment policy: The value for the variable is used regardless of whether or not the intercurrent event occurs.
^{b} Hypothetical: A scenario is envisioned in which the intercurrent event would not occur.
^{c} Composite: Incorporation of intercurrent events in the definition of the endpoint.

### 7.3.2.3 Mean Score Estimand and Estimator

The elements of the mean score estimand are defined in Table 6. The comparison of interest is the difference between the placebo and satralizumab groups in the change in CASE score from baseline at Week 24, regardless of SDCR withdrawal from study treatment, treatment interruption or change in background therapy not considered rescue, assuming rescue therapy had not been available, and assuming participants with NSDCR withdrawals would have continued to receive their randomized treatment.

TABLE 6
Attributes of Mean Change Estimand

| | |
|---|---|
| Population | Participants with NMDAR or LGI1 encephalitis, as defined |
| | in Section 4.1 |
| Variable or endpoint | CASE score |
| Treatment | Satralizumab versus matching placebo, administered for |
| | 52 weeks, in combination with background therapy |
| Intercurrent events | |
| SDCR withdrawal from study | Treatment policy ^{a} |
| treatment | |
| NSDCR withdrawal from study | Hypothetical ^{b} |
| treatment | |
| Death | Assume worst value |
| Taking rescue therapy | Hypothetical ^{b} |
| Treatment interruption (e.g., due | Treatment policy ^{a} |
| to infection) | |
| Change in background therapy | Treatment policy ^{a} |
| not considered rescue | |
| Summary measure | The difference in the mean change from baseline to |
| | Week 24 in CASE score |
| Method of estimation | An estimate of the treatment effect will be computed using |
| | an MMRM analysis adjusting for the randomization |
| | stratification factors and baseline score. |
| | Missing data following NSDCR and SDCR withdrawals will |
| | be imputed as described for the primary endpoint |
| | (Section 7.3.1.2). For the intercurrent event of taking |
| | rescue therapy, any data collected after the initiation of |
| | rescue therapy will be censored. Data following this |
| | intercurrent event will be imputed using reference based |
| | imputation with a “Copy Reference” assumption. |

CASE = Clinical Assessment Scale of Encephalitis; LGI1 = leucine-rich glioma in-activated 1; MMRM = mixed model repeated measures; NMDAR = N-methyl-D-aspartic acid receptor; NSDCR = not study drug or condition related; SDCR = study drug or condition related.
^{a} Treatment policy: The value for the variable is used regardless of whether or not the intercurrent event occurs.
^{b} Hypothetical: A scenario is envisioned in which the intercurrent event would not occur.

### 7.3.2.3 Mean Score Estimand and Estimator

The elements of the mean score estimand are defined in Table 7 using the MOCA total score endpoint as an example. The comparison of interest is the difference between the placebo and satralizumab groups in the MOCA total score at Week 24, regardless of SDCR withdrawal from study treatment, treatment interruption or change in background therapy not considered rescue, assuming rescue therapy had not been available, and assuming participants with NSDCR withdrawals would have continued to receive their randomized treatment.

TABLE 7
Attributes of Mean Score Estimand

| | |
|---|---|
| Population | Participants with NMDAR or LGI1 encephalitis, as defined |
| | in Section 4.1 |
| Variable or endpoint | MOCA total score |
| Treatment | Satralizumab versus matching placebo, administered for |
| | 52 weeks, in combination with background therapy |
| Intercurrent events | |
| SDCR withdrawal from study | Treatment policy ^{a} |
| treatment | |
| NSDCR withdrawal from study | Hypothetical ^{b} |
| treatment | |
| Death | Assume worst value |
| Taking rescue therapy | Hypothetical ^{b} |
| Treatment interruption (e.g., due | Treatment policy ^{a} |
| to infection) | |
| Change in background therapy | Treatment policy ^{a} |
| not considered rescue | |
| Summary measure | The difference in the mean MOCA total score at Week 24 |
| Method of estimation | An estimate of the treatment effect will be computed using |
| | an MMRM analysis adjusting for the randomization |
| | stratification factors and baseline score. |
| | Missing data following NSDCR and SDCR withdrawals will |
| | be imputed as described for the primary endpoint |
| | (Section 7.3.1.2). For the intercurrent event of taking |
| | rescue therapy, any data collected after the initiation of |
| | rescue therapy will be censored. Data following this |
| | intercurrent event will be imputed using reference based |
| | imputation with a “Copy Reference” assumption. |

LGI1 = leucine-rich glioma inactivated 1; MOCA = Montreal Cognitive Assessment; MMRM = mixed model repeated measures; NMDAR = N-methyl-D-aspartic acid receptor; NSDCR = not study drug or condition related; SDCR = study drug or condition related.
^{a} Treatment policy: The value for the variable is used regardless of whether or not the intercurrent event occurs.
^{b} Hypothetical: A scenario is envisioned in which the intercurrent event would not occur.

### 7.3.2.4 Categorical Score Estimand and Estimator

The elements of the categorical score estimand are defined in Table 8. The comparison of interest is the difference between the placebo and satralizumab groups in the mRS score at Week 24, regardless of SDCR withdrawal from study treatment, treatment interruption or change in background therapy not considered rescue, assuming rescue therapy had not been available, and assuming participants with NSDCR withdrawals would have continued to receive their randomized treatment.

TABLE 8
Attributes of Categorical Score Estimand

| | |
|---|---|
| Population | Participants with NMDAR or LGI1 encephalitis, as defined |
| | in Section 4.1 |
| Variable or endpoint | mRS score |
| Treatment | Satralizumab versus matching placebo, administered for |
| | 52 weeks, in combination with background therapy |
| Intercurrent events | |
| SDCR withdrawal from study | Treatment policy ^{a} |
| treatment | |
| NSDCR withdrawal from study | Hypothetical ^{b} |
| treatment | |
| Taking rescue therapy | Hypothetical ^{b} |
| Treatment interruption (e.g., due | Treatment policy ^{a} |
| to infection) | |
| Change in background therapy | Treatment policy ^{a} |
| not considered rescue | |
| Summary measure | Common odds ratio at Week 24 |
| | An estimate of the treatment effect will be computed using |
| | a proportional odds model adjusting for the randomization |
| | stratification factors. The p-value will be calculated using a |
| | Van Elteren test, including the stratification factors at |
| | randomization. |
| Method of estimation | Missing data following NSDCR and SDCR withdrawals will |
| | be imputed as described for the primary endpoint |
| | (Section 7.3.1.2). For the intercurrent event of taking |
| | rescue therapy, any data collected after the initiation of |
| | rescue therapy will be censored. Data following this |
| | intercurrent event will be imputed using reference based |
| | imputation with a “Copy Reference” assumption. |

LGI1 = leucine-rich glioma inactivated 1; mRS = Modified Rankin Scale; NMDAR = N-methyl-D-aspartic acid receptor; NSDCR = not study drug or condition related; SDCR = study drug or condition related.
^{a} Treatment policy: The value for the variable is used regardless of whether or not the intercurrent event occurs.
^{b} Hypothetical: A scenario is envisioned in which the intercurrent event would not occur.

### 7.3.2.5 Safety Analysis

Safety will be assessed through summaries of exposure to study treatment, adverse events, changes in laboratory test results, and changes in vital signs, weight, height (<18 years only), and ECGs.

Study treatment exposure (such as treatment duration, total dose received, and dose modifications) will be summarized with descriptive statistics.

All verbatim adverse event terms will be mapped to Medical Dictionary for Regulatory Activities thesaurus terms, and adverse event severity will be graded according to National Cancer Institute (NCI) Common Terminology Criteria for Adverse Events (CTCAE) v5.0. All adverse events, serious adverse events, adverse events leading to death, adverse events of special interest, and adverse events leading to study treatment discontinuation that occur on or after the first dose of study treatment (i.e., treatment-emergent adverse events) will be summarized by mapped term, appropriate thesaurus level, and severity grade. For events of varying severity, the highest grade will be used in the summaries. Deaths and cause of death will be summarized.

Relevant laboratory, vital sign (pulse rate, respiratory rate, blood pressure, pulse oximetry, and temperature), and ECG data will be displayed by time, with grades identified where appropriate. Additionally, a shift table of selected laboratory tests will be used to summarize the baseline and maximum postbaseline severity grade. Changes in vital signs, weight, height (<18 years only), and ECGs will be summarized.

### 7.3.3 Exploratory Endpoints

Subgroup analyses will be performed for the following populations:
Adolescent participants with NMDAR encephalitis
Participants with probable NMDAR encephalitis

More details on the definition and analysis of exploratory endpoints will be given in the SAP.

### 7.3.4 Other Analyses

### 7.3.4.1 Pharmacokinetic Analyses

The PK analysis population consists of all participants in the safety analysis set with at least one valid postdose concentration result with a dosing record and sampling time. The trial will evaluate the PK characteristics of satralizumab treatment by summary statistics and non-linear mixed effects analysis (popPK).

Both the satralizumab concentration data and the results of the popPK analysis will be reported separately from the CSR.

### 7.3.4.2 Immunogenicity Analyses

The immunogenicity analysis population will consist of all participants with at least one ADA assessment. Participants will be grouped according to treatment received or, if no treatment is received prior to study discontinuation, according to treatment assigned.

The numbers and proportions of ADA-positive participants and ADA-negative participants at baseline (baseline prevalence) and after drug administration (postbaseline incidence) will be summarized by treatment group. When determining postbaseline incidence, participants are considered to be ADA-positive if they show treatment-induced ADA response or treatment-enhanced ADA response. Participants who are ADA-negative or have missing data at baseline, but develop an ADA response following study drug exposure have a treatment-induced ADA response. Participants who are ADA-positive at baseline and the titer of one or more postbaseline samples is at least 4-fold (0.60 titer unit) greater than the titer of the baseline sample have a treatment-enhanced ADA response. Participants are considered to be ADA-negative if they are ADA-negative or have missing data at baseline and all postbaseline samples are negative, or if they are ADA-positive at baseline but do not have any postbaseline samples with a titer that is at least 4-fold (0.60 titer unit) greater than the titer of the baseline sample (treatment unaffected).

The percentage of participants who have positive or negative ADA results for satralizumab will be tabulated. PK, PD, efficacy parameters, and safety will be summarized by anti-satralizumab antibody (i.e., satralizumab ADA) status.

### 7.3.4.3 Pharmacodynamic Analyses

Serum IL-6 and sIL-6R levels will be summarized by treatment group and timepoint graphically and descriptively, as appropriate.

7.4 interim analyses

### 7.4.1 Planned Interim Pharmacokinetic Analyses

An interim PK analysis will be performed at Week 8 of treatment, with the purpose of confirming that the achieved exposures (and predicted RO) are within the expected target range. If the achieved exposures (and predicted RO) are not within the expected target range the doses may be adapted to 120 mg, 180 mg, and 240 mg for participants <40 kg, between 40 and 100 kg (inclusive), and >100 kg, respectively. The chosen dosing regimen will be associated with exposures that do not significantly exceed the existing exposure-safety coverage.

The iDMC will make a recommendation on whether (i) the trial can be continued with the initial dose or whether (ii) the dose can be adapted to the pre-specified higher doses.

### 7.4.2 Optional Interim Analysis

To adapt to information that may emerge during the course of this study, the Sponsor may choose to conduct one interim futility analysis. Below are the specifications to ensure the study continues to meet the highest standards of integrity when an optional interim analysis is executed.

If an interim analysis is conducted, the Sponsor will remain blinded. The interim analysis will be conducted by an external statistical group and reviewed by the iDMC. Interactions between the iDMC and Sponsor will be carried out as specified in the iDMC Charter.

The decision to conduct the optional interim analysis, along with the rationale, timing, and statistical details for the analysis will be documented in the SAP. The iDMC Charter will be updated to document potential recommendations the iDMC can make to the Sponsor as a result of the analysis (continue the study without modification, stop the study for futility), and the iDMC Charter will also be made available to relevant health authorities.

If there is a potential for the study to be stopped for futility as a result of the interim analysis, the threshold for declaring futility may include an assessment of the predictive probability that the specified endpoint will achieve statistical significance. Criteria for recommending that the study be stopped for futility may be added to the iDMC Charter and will be documented in the SAP.

### INDUSTRIAL APPLICABILITY

The present invention provides a means for a treatment for an autoimmune encephalitis, such as NMDAR or LGI1 encephalitis, and also for a reduction of a risk of relapsing said encephalitis, comprising an anti-IL-6 receptor antibody or antigen binding fragment thereof. The present invention also provides a medicament or a pharmaceutical composition for a treatment of or a reduction of a risk of relapsing said encephalitis comprising an anti-IL-6 receptor antibody or antigen binding fragment thereof. The present invention further provides a method of a treatment of or a reduction of a risk of relapsing said encephalitis by administering an anti-IL-6 receptor antibody or antigen binding fragment thereof to a subject in need thereof.

### REFERENCES

Abboud H, Probasco J, Irani S R, et al. Autoimmune encephalitis: proposed recommendations for symptomatic and long-term management. J Neurol Neurosurg Psychiatry 2021. doi: 10.1136/jnnp-2020-325302.
Archarya J N, Hani A, Thirumala P et al. Guideline 3: A proposal for standard montages to be used in clinical EEG. Am Clin Neurophysiol Soc 2016:1-16.
Armangue T, Spatola M, Vlagea A, et al. Frequency, symptoms, risk factors, and outcomes of autoimmune encephalitis after herpes simplex encephalitis: a prospective observational study and retrospective analysis. Lancet Neurol 2018; 17:760-72.
Banks J L, Marotta C A. Outcomes validity and reliability of the modified Rankin scale: implications for stroke clinical trials: a literature review and synthesis. Stroke 2007; 38:1091-6.
Beck A T, Ward C H, Mendelson M, et al. An inventory for measuring depression. Arch Gen Psychiatry 1961; 4: 561-71.
Bien C G, Vincent A, Barnett M H, et al. Immunopathology of autoantibody-associated encephalitides: clues for pathogenesis. Brain 2012 May; 135(Pt 5):1622-38. Brooks R. EuroQol: the current state of play. Health Policy 1996; 37:53-72. Cadena R, Shoykhet M, Ratcliff J. Emergency neurological life support: intracranial hypertension and herniation. Neurocrit Care 2017; 27(Suppl 1):82-8.
Dalmau J, Armangue T, Planaguma J, et al. An update on anti-NMDA receptor encephalitis for neurologists and psychiatrists: mechanisms and models. Lancet Neurology 2019; 11:1045-1057.
Ding Y-W, Pan S-Y, Xie W, et al. Elevated soluble fas and fasL in cerebrospinal fluid and serum of patients with anti-N-methyl-D-aspartate receptor encephalitis. Front Neurol 2018 9:904.
Dubey D, Pittock S, Kelly C, et al. Autoimmune encephalitis epidemiology and a comparison to infectious encephalitis. Ann Neurol 2018; 83:166-77.
Dubey D, Sawhney A, Greenberg B, et al. The spectrum of autoimmune encephalopathies. J Neuroimmunol 2015; 15:93-7.
EuroQol Group. EuroQol: a new facility for the measurement of health-related quality of life. Health Policy 1990; 16:199-208.
Food and Drug Administration. Guidance for industry, “Enrichment strategies for clinical trials to support approval of human drugs and biological products” [March 2019]. Available from: https://www.fda.gov/regulatory-information/search-fda-guidance-documents/enrichme nt-strategies-clinical-trials-support-approval-human-drugs-and-biological-products.
Fisk J D, Pontefract A, Ritvo P G, et al. The impact of fatigue on patients with multiple sclerosis. Can J Neurol Sci 1994a; 21:9-14.
Fisk J D, Ritvo P G, Ross L, et al. Measuring the functional impact of fatigue: initial validation of the Fatigue Impact Scale. Clin Infect Dis 1994b; 18(Supplement_1):S79-83.
Gadoth A, Pittock S, Dubey D, et al. Expanded phenotypes and outcomes among 256 LGI1/CASPR2-IgG-positive patients. Ann Neurol. 2017; 82:79-92.
Gadoth A, Zekeridou A, Klein C J, et al. Elevated LGI1-IgG CSF index predicts worse neurological outcome. Ann Clin Transl Neurol 2018; 5:646-50.
Graus G, Titulaer M, Benseler S, et al. A clinical approach to diagnosis of autoimmune encephalitis. Lancet Neurol 2016; 15:391-404.
Gruter T, Ott A, Meyer W, et al. Effects of IVIg treatment on autoantibody testing in neurological patients: marked reduction in sensitivity by reliable specificity. J Neurology 2020; 267(1).
Hang H L, Zhang J H, Chen D W, et alP. Clinical characteristics of cognitive impairment and 1-year outcome in patients with anti-LGI1 antibody encephalitis. Front Neurol 2020; 11:852.
Harrison J K, McArthur K S, Quinn T J. Assessment scales in stroke: clinimetric and clinical considerations. Clin Interv Aging 2013; 8:201-11.
Helmstaedter C, Hansen N, Leelaarporn P, et al. Specific B- and T-cell populations are associated with cognition in patients with epilepsy and antibody positive and negative suspected limbic encephalitis. J Neurol 2021; 268:455-66.
Herdman M, Gudex C, Lloyd A, et al. Development and preliminary testing of the new five-level version of EQ-5D (EQ-5D-5L). Qual Life Res 2011; 20:1727-36.
Ho ACC, Mohammad S S, Pillai S C, et al. High sensitivity and specificity in proposed clinical diagnostic criteria for anti-N-methyl-D-aspartate receptor encephalitis. Dev Med Child Neurol 2017; 59:1256-60.
Hughes E G, Peng X, Gleichman A J, et al. Cellular and synaptic mechanisms of anti-NMDA receptor encephalitis. J Neurosci 2010; 30:5866-75.
Janssen M F, Pickard A S, Golicki D, et al. Measurement properties of the EQ-5D-5L compared to the EQ-5D-3L across eight patient groups: a multi-country study. Qual Life Res 2013; 22:1717-27.
Johns M W. A new method for measuring daytime sleepiness: the Epworth Scale. Sleep 1991; 14:540-5.
Jun J-S, Lee S-T, Kim R, et al. Tocilizumab treatment for early treatment refractory status epilepticus. Ann Neurol 2018; 84:940-5.
Kaneko A, Kaneko J, Tominaga N et al. Pitfalls in clinical diagnosis of anti-NMDA receptor encephalitis. J Neurol 2018 March; 265:586-96.
Kirmani B, Barr D, Robinson D M, et al. Management of autoimmune status epilepticus. Front Neurol 2018; 9:259.
Kluger B M, Krupp L B, Enoka R M. Fatigue and fatigability in neurologic illnesses: proposal for a unified taxonomy. Neurology 2013; 80:409-16.
Krogias C, Hoepner R, Muller A, et al. Successful treatment of anti-Caspr2 syndrome by interleukin 6 receptor blockade through tocilizumab. JAMA Neurol 2013; 70: 1056-9.
Lee W J, Lee S T, Byun J I, et al. Rituximab treatment for autoimmune limbic encephalitis in an institutional cohort. Neurology 2016a; 86:1683-91.
Lee W J, Lee S T, Moon J, et al. Tocilizumab in autoimmune encephalitis refractory to rituximab: an institutional cohort study. Neurotherapeutics 2016b; 13:824-32.
Lee W J, Lee S T, Shin Y W, et al. Teratoma removal, steroid, IVIG, rituximab and tocilizumab (T-SIRT) in anti-NMDAR encephalitis. Neurotherapeutics 2020; doi.org/10.1007/s13311-020-00921-7.
Leypoldt F, Hoftberger R, Titulaer M J, et al. Investigations on CXCL13 in anti-N-methyl-D-aspartate receptor encephalitis: a potential biomarker of treatment response. JAMA Neurol. 2015; 72:180-6.
Leypoldt F, Wandinger K-P, Bien C, et al. Autoimmune encephalitis. Eur Neurol Rev 2013; 8:31-7.
Lim J A, Lee S T, Moon J. Development of the clinical assessment scale in autoimmune encephalitis. Ann Neurol 2019; 85:352-8.
Martinez-Hernandez E, Horvath J, Shiloh-Malawsky Y, et al. Analysis of complement and plasma cells in the brain of patients with anti-NMDAR encephalitis. Neurology 2011; 7:589-93.
Mikasova L, De Rossi P, Bouchet D, et al. Disrupted surface cross-talk between NMDA and Ephrin-B2 receptors in anti-NMDA encephalitis. Brain 2012; 135:1606-21.
Moscato E H, Peng X, Jain A, et al. Acute mechanisms underlying antibody effects in anti-N-methyl-D-aspartate receptor encephalitis. Ann Neurol 2014; 76:108-19.
Nishida H, Kohyama K, Kumada S, et al. Evaluation of the diagnostic criteria for anti-NMDA receptor encephalitis in Japanese children. Neurology 2021; 96:e2070-e2077.
Ohkawa T, Fukata Y, Miwako Y, et al. Autoantibodies to epilepsy-related LGI1 in limbic encephalitis neutralize LGI1-ADAM22 interaction and reduce synaptic AMPA receptors. J Neurosci 2013; 33:18161-74.
Peaker A, Stewart L E. Rey's Auditory-Verbal Learning Test-a review. Dev Clin Exper Neuropsychology 1989:219-36.
Petit-Pedrol M, Sell J, Planaguma J, et al. LGI1 antibodies alter Kv1.1 and AMPA receptors changing synaptic excitability, plasticity and memory. Brain 2018; 141:3144-59.
Randell R, Adams A, Van Mater H. Tocilizumab in refractory autoimmune encephalitis: a series of pediatric cases. Ped Neurology 2018; 86:66-8. Rankin J. Cerebral vascular accidents in patients over the age of 60: II. Prognosis. Scott Med J 1957; 2:200-15.
Rosch R E, Wright S, Cooray G, et al. NMDA-receptor antibodies alter cortical microcircuit dynamics. Proc Natl Acad Sci USA 2018; 115:E9916-25.
Shin Y-W, Lee S-T, Park K-I, et al. Treatment strategies for autoimmune encephalitis. Ther Adv Neurol Disord 2018; 11:1756285617722347.
Spreen O, Strauss E. A compendium of neuropsychological tests: administration, norms, and commentary. New York: Oxford University Press, 1991.
Titulaer M, McCracken L, Gabilondo I, et al. Treatment and prognostic factors for long-term outcome in patients with anti-NMDA receptor encephalitis: an observational cohort study. Lancet Neurol 2013; 12:157-65.
van Sonderen A, Thijs R, Coenders E, et al. Anti-LGI1 encephalitis: clinical syndrome and long-term follow-up. Neurology 2016; 87:1449-56.
Venkatesan A, Tunkel A R, Bloch K C, et al. Case definitions, diagnostic algorithms, and priorities in encephalitis: consensus statement of the International Encephalitis Consortium. Clin Infect Dis 2013; 57:1114-28.
Wang X, Ma C, Liu C Y et al. Neuronal NMDAR currents of the hippocampus and learning performance in autoimmune anti-NMDAR encephalitis and involvement of TNF-alpha and IL-6. Front Neurol 2019; 10:684.
Wilson J L, Hareendran A, Hendry A, et al. Reliability of the modified Rankin Scale across multiple raters: benefits of a structured interview. Stroke 2005; 36:777-81.

1. A medicament for treating an autoimmune encephalitis (AIE) in a subject, comprising satralizumab.

2. The medicament of claim 1, for delaying relapse of, reducing frequency of relapse of, reducing severity of relapse of, or reducing risk of relapse of the autoimmune encephalitis.

3. The medicament of claim 1 or 2, wherein the autoimmune encephalitis is anti-N methyl-D aspartic acid receptor (NMDAR) encephalitis or anti-leucine rich glioma inactivated 1 (LGI1) encephalitis.

4. The medicament of any one of claims 1-3, wherein the medicament is administered to the subject subcutaneously.

5. The medicament of any one of claims 1-4, wherein the medicament provides a 60 mg, 120 mg, 180 mg, or 240 mg dosage unit of satralizumab to the subject.

6. The medicament of any one of claims 1-5, wherein the medicament is used such that 60 mg of satralizumab is administered to the subject with body weight of less than 40 kg for each administration.

7. The medicament of any one of claims 1-5, wherein the medicament is used such that 120 mg of satralizumab is administered to the subject with body weight of less than 40 kg for each administration.

8. The medicament of any one of claims 1-5, wherein the medicament is used such that 120 mg of satralizumab is administered to the subject with body weight of between 40 and 100 kg for each administration.

9. The medicament of any one of claims 1-5, wherein the medicament is used such that 180 mg of satralizumab is administered to the subject with body weight of between 40 and 100 kg for each administration.

10. The medicament of any one of claims 1-5, wherein the medicament is used such that 180 mg of satralizumab is administered to the subject with body weight of over 100 kg for each administration.

11. The medicament of any one of claims 1-5, wherein the medicament is used such that 240 mg of satralizumab is administered to the subject with body weight of over 100 kg for each administration.

12. The medicament of any one of claims 1-11, wherein the medicament is administered to the subject every two weeks (Q2W) for three times, and thereafter every four weeks (Q4W).

13. The medicament of any one of claims 1-12, wherein the medicament is used in combination with an immunosuppressive therapy (TST).

14. The medicament of claim 13, wherein the IST is a therapy with (i) an immunosuppressive agent selected from the group consisting of azathioprine (AZA), mycophenolate mofetil (MMF), and intravenous (IV) cyclophosphamide; (ii) oral corticosteroid (OCS); or (iii) a combination of (i) and (ii).

15. The medicament of claim 14, wherein the immunosuppressive agent comprises prednisone, prednisolone or equivalent thereof.

16. The medicament of claim 5 which is administered by a subcutaneous administration device.

17. The medicament of claim 16, wherein the the subcutaneous administration device is a pre-filled syringe (PFS) optionally comprising a needle safety device (PFS-NSD) or autoinjector (AT).
